(19) United States
(12) Patent Application Publication
(10) Pub. No.: US 2024/0257912 A1
(43) Pub. Date: Aug. 1, 2024

(54) Scalable Distributed Processing Software for Next-Generation in Situ Sequencing

(71) Applicant: The Board of Trustees of the Leland Stanford Junior University, Stanford, CA (US)

(72) Inventors: Karl A. Deisseroth, Stanford, CA (US); Ethan B. Richman, Palo Alto, CA (US); Christopher M. Roat, Redwood City, CA (US)

(21) Appl. No.: 18/561,643

(22) PCT Filed: May 20, 2022
(86) PCT No.: PCT/US2022/030321

§ 371 Date: Nov. 16, 2023

Related U.S. Application Data
(60) Provisional application No. 63/191,462, filed on May 21, 2021.

(51) Int. Cl. G16B 30/00; G06T 7/00; G06T 7/33; G06V 20/69

(52) U.S. Cl. CPC G16B 30/00; G06T 7/0016; G06T 7/337; G06V 20/695; G06V 20/698; G06T 2207/10024; G06T 2207/10064; G06T 2207/20092; G06T 2207/30024; G06T 2207/30242

(57) ABSTRACT

Methods, systems, and devices, including computer programs encoded on a computer storage medium are provided for processing of next-generation in situ sequencing data for nucleic acids in cells in tissue. In particular, cloud-based scalable data processing software for volumetric in situ sequencing is provided.

### BACKGROUND OF THE INVENTION

Biological samples contain complex and heterogenous genetic information spanning the length scales of individual cells and whole tissues. Spatial patterns of nucleic acids within a cell may reveal properties and abnormalities of cellular function; cumulative distributions of RNA expression may define a cell type or function; and systematic variation in the locations of cell types within a tissue may define tissue function. The combination of anatomical connectivity information encoded in nucleic acids and tissue-wide cell type distributions may span many sections of tissue. Techniques for in situ nucleic acid sequencing must therefore be able to bridge resolutions as small as individual molecules and as large as entire brains. Efficiently collecting and recording this information across orders-of-magnitude differences in lengths requires novel inventions to enhance the robustness, rapidity, automated-, and high throughput-nature of in situ sequencing techniques.

### SUMMARY OF THE INVENTION

Methods, systems, and devices, including computer programs encoded on a computer storage medium are provided for processing of next-generation in situ sequencing data for nucleic acids in cells in tissue. In particular, cloud-based scalable data processing software for volumetric in situ sequencing is provided.

In one aspect, a computer implemented method for processing in situ sequencing imaging data is provided, the computer performing steps comprising: (a) receiving in situ sequencing imaging data; (b) applying configuration parameters to the in situ sequencing imaging data, wherein the configuration parameters comprise an encoding scheme, a codebook, image acquisition parameters, and sample metadata; (c) preprocessing imaging data by removing optical aberrations, subtracting background signal, performing deconvolution, filtering the images, and merging overlapping fields of view; (d) performing registration of imaging data by aligning selected common features in images taken at different timepoints and across different color channels at each time point; (e) segmenting images to determine locations of cell nuclei and cells; and (f) performing sequential analysis of images if the in situ sequencing imaging data is from sequential sequencing, wherein said performing sequential analysis of images comprises: i) measuring an intensity of a fluorescent signal for each segmented cell and nucleus, wherein the intensity of the fluorescence signal is proportional to the amount of a target nucleic acid, and ii) subtracting estimated carry-over signal from a previous sequencing round, or performing combinatorial analysis of images if the in situ sequencing imaging data is from combinatorial sequencing, wherein said performing sequential analysis of images comprises i) identifying each amplicon labeling a target nucleic acid from detection of barcodes, wherein invalid barcodes are removed according to their encoding scheme, ii) measuring a fluorescent signal of each amplicon at each timepoint to determine which color channel has the highest signal for each detected amplicon, closest code-book entry to a vector of signals, or clustering of pixel intensities; iii) iteratively matching signals across rounds of sequencing to membership in the code-book; iv) assigning a target identity to each detected amplicon, and v) calculating the number of each target nucleic acid present in each cell and nucleus.

In certain embodiments, the in situ sequencing data are stored in a cloud data storage system. In some embodiments, the cloud data storage system is a public cloud storage system or a private cloud storage system.

In certain embodiments, the configuration parameters are provided by a configuration file. In other embodiments, the configuration parameters are provided by a subject inputting the configuration parameters using a management web interface.

In certain embodiments the imaging data comprises morphology images, images from sequential sequencing, or images from combinatorial sequencing.

In certain embodiments, the method further comprises optimizing processing parameters by performing multiple rounds of processing of the in situ sequencing imaging data by reiterating steps (a)-(f), wherein different configuration parameters are used to process the in situ sequencing imaging data for each round of data processing.

In certain embodiments, the imaging data comprises images taken at multiple timepoints.

In certain embodiments, the imaging data comprises images from multiple color channels at each time point.

In certain embodiments, the imaging data further comprises morphological information, sequential readout amplicon data, or a single base of combinatorial readout amplicon data.

In certain embodiments, performing registration comprises aligning images based on detection of a common imaging dye, detectably labeled antibody, or chemical label used to stain a cell component. In some embodiments, the common imaging dye is a DNA dye used to stain nuclei. In some embodiments, the DNA dyes is a fluorescent DNA dye. An exemplary DNA dye includes, without limitation, 4′,6-diamidino-2-phenylindole (DAPI).

In certain embodiments, performing registration comprises aligning images based on detection of immunofluorescence staining of a cell component. In some embodiments, the cell component is a cell membrane marker.

In certain embodiments, performing registration comprises aligning images based on detection of fluorescently labeled amplicons. In some embodiments, performing registration comprises aligning images taken at each time point based on detection of a fluorophore used to label amplicons. In some embodiments, the method further comprises aligning images from combinatorial sequencing taken at different times or from different color channels.

In certain embodiments, performing registration of imaging data from combinatorial sequencing comprises performing intra-channel registration to generate intra-channel registered data; and performing inter-channel registration on the intra-channel registered data. In some embodiments, overlap in amplicon features is used to align images for intra-channel registration at a sub-pixel level. In some embodiments, inter-channel registration comprises performing a roundmax-projection on the intra-channel registered data.

In certain embodiments, the method further comprises outputting aligned images of all amplicons appearing in a color channel for each channel for each time point.

In certain embodiments, segmenting comprises segmenting images based on detecting cell nuclei. In some embodiments, segmenting further comprises segmenting images based on detecting cells.

In certain embodiments for sequential sequencing, estimating carry-over signal comprises identifying isolated amplicons for a given color channel and round of sequencing, measuring a fluorescent signal for each isolated amplicon across subsequent time points in all color channels, and selecting the isolated amplicon having the brightest fluorescent signal to measure median carry-over fraction from a given detection time point to subsequent measurement time points. In some embodiments, the method further comprises correcting fluorescent signals for a cell by calculating the true signal (S_true) based on the measured signal (S_measured) and the carry over matrix (M_co) according to the equation: S_measured=S_true*M_co.

In certain embodiments, imaging data is divided into chunks for processing by a plurality of graphics processing units (GPUs), field-programmable gate arrays (FPGAs), or tensor processing units (TPUs).

In certain embodiments, segmenting images further comprises performing distributed stitching segmentation to stitch together cells that cross chunk boundaries.

In certain embodiments, the method further comprises displaying a target identity and target location for each identified target nucleic acid superimposed on a processed image of the tissue. In some embodiments, the method further comprises displaying the number of each target nucleic acid present in each cell and nucleus.

In certain embodiments, a computer implemented method is provided, the method comprising: detecting amplicon locations by a method comprising: choosing a channel with the maximum signal per round of sequencing for each amplicon; finding barcodes of the amplicons in an encoding dictionary; applying error correction; performing cross-channel normalization; summing signals per channel per round per amplicon; performing probabilistic modeling of each amplicon based on the summing signals per channel per amplicon, background signals, and carry-over signals per sequencing round; and decoding sources of signals using posterior likelihood probabilities with regularization or sparsity constraints.

In certain embodiments, a computer implemented method is provided, the method comprising: performing a matching pursuit, an orthogonal matching pursuit, or a compressed sensing regime for matrix decomposition of pixel channel-round-intensity matrices into barcode-presence indicator vectors, barcode dictionary channel-round matrices, estimated background, estimated channel bleed through, and estimated channel carryover matrices; identifying a location of an amplicon by detecting barcode membership vectors in pixel space; performing probabilistic modeling of the image channel-round volume with both the number and x, y, and z coordinates of sources; converting each pixel to a signal per a channel-round matrix; assigning the corresponding barcode dictionary channel-round signal matrix entry based on a nearest neighbor; and locating and counting assigned amplicons.

In another aspect, a non-transitory computer-readable medium comprising program instructions that, when executed by a processor in a computer, causes the processor to perform a computer implemented method for processing in situ sequencing imaging data, described herein, is provided.

In another aspect, a kit comprising the non-transitory computer-readable medium described herein and instructions for processing in situ sequencing imaging data is provided.

In another aspect, a system is provided, the system comprising: a processor programmed to process in situ sequencing imaging data of target nucleic acids in a tissue according to a computer implemented method described herein; and a display component for displaying information regarding the processed in situ sequencing imaging data.

In certain embodiments, the processor is provided by a computer or handheld device (e.g., a cell phone or tablet). In some embodiments, the processor is provided by a cloud computer.

In certain embodiments, the system further comprises a hardware accelerator.

In certain embodiments, the system further comprises a plurality of graphics processing units (GPUs), field-programmable gate arrays (FPGAs), or tensor processing units (TPUs).

In certain embodiments, the display component displays information regarding the sequential analysis of images if the in situ sequencing imaging data is from sequential sequencing, or displays information regarding the combinatorial analysis of images if the in situ sequencing imaging data is from combinatorial sequencing.

In certain embodiments, the display component displays a target identity and target location for each identified target nucleic acid superimposed on a processed image of the tissue.

In certain embodiments, the display component displays the number of each target nucleic acid present in each cell and nucleus.

In certain embodiments, the system further comprises a storage component. In some embodiments, the storage component is cloud storage.

In certain embodiments, the system further comprises a sequencer for performing in situ sequencing.

In certain embodiments, the system further comprises reagents for performing in situ sequencing.

In certain embodiments, the system further comprises agents for performing image registration or image segmentation.

In certain embodiments, the system further comprises an imaging chamber.

In another aspect, a kit comprising a system described herein and instructions for processing in situ sequencing imaging data is provided.

### BRIEF DESCRIPTION OF THE DRAWINGS

FIG. 1 shows a schematic overview of the data processing software for next-generation in situ sequencing. Upon acquisition, data is streamed to cloud storage, along with a configuration file specifying the encoding scheme, codebook, and image acquisition and sample metadata. Data arriving in the cloud is entered into the cloud processing pipeline as specified by the configuration file. This custom cloud processing pipeline, is deployed on top of Kubernetes, a cloud provider-agnostic platform. Cloud data storage is a core component, and houses the raw, intermediate, and final data products. Processing begins with the upload of a dataset into the storage by the data acquisition system. Pipeline configuration parameters are set by the scientist in a data management web interface or generated automatically from a configuration file uploaded with the sequencing data. Each configuration is stored in a DataJoint cloud database; multiple configurations can be applied to a single dataset, allowing optimization of processing parameters using multiple processing runs. A set of data processing workers query the database for any outstanding work, perform the work, and inform the database as work is completed. Work requiring multiple computers is farmed out to a Dask cluster which autoscales to perform the work quickly. A single dataset can create multiple terabytes of intermediate data in cloud storage; this data can be efficiently visualized using Neuroglancer or analyzed in JupyterHub.

FIG. 2 shows processing of morphology images, sequential images, and combinatorial images. The image processing platform transforms raw microscope images into cell locations, 3D locations and molecular identities of amplicons via combinatorial readout, and per-cell amplicon signal via sequential readout. A round may include native or other fluorescence, morphological information, sequentially readout amplicon data, or a single base of combinatorial readout amplicon data. Data is immediately transformed by two steps: a preprocessing stage that removes chromatic aberration and performs background removal and deconvolution; and a stitching step that merges overlapping fields of view together. Once all rounds are preprocessed and stitched, two registration steps are performed in parallel: a large-scale registration using DAPI stain collected in each round, and a more precise amplicon registration using a multi-round combined fit to correct for camera offsets, residual chromatic effects, and other artefacts. Next, nuclei and cells are segmented using the morphological data (a fluorophore-labeled hybridization sequence complementary to the unique sequence of the oligo-dT label, yielding a proxy signal in the hydrogel of the total mRNA location). Amplicons are detected, decoded, and assigned to cells. In parallel, the sequential data goes through post-processing and target levels (amplicon signal of a particular encoding) are estimated per-cell.

FIG. 3 shows processing of raw or compressed sequencing data for a sample run.

FIG. 4 shows the transformation of the raw imaging data (e.g., in tiff files) to fully chunked and distributed monolithic datastores for optimal parallel/distributed access and downstream processing.

FIG. 5 shows combinatorial featurization approaches.

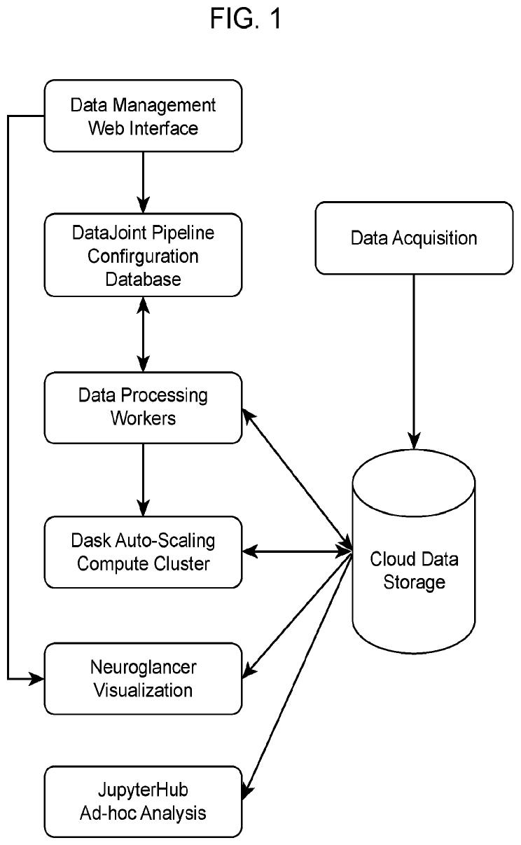

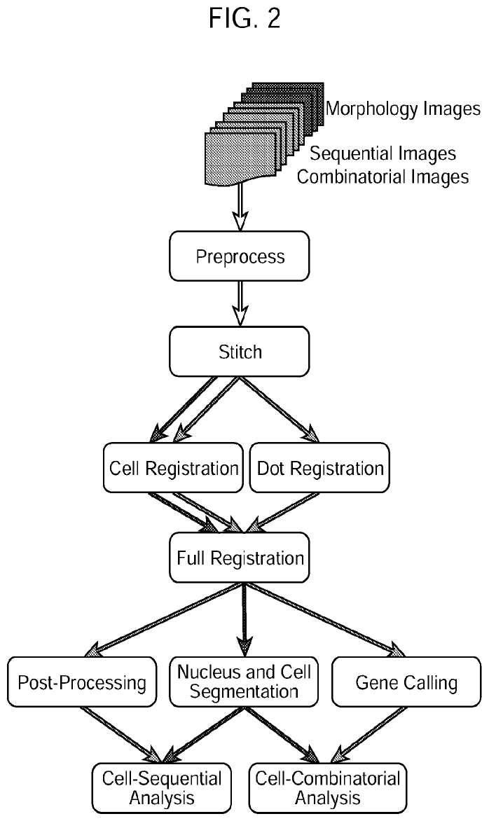

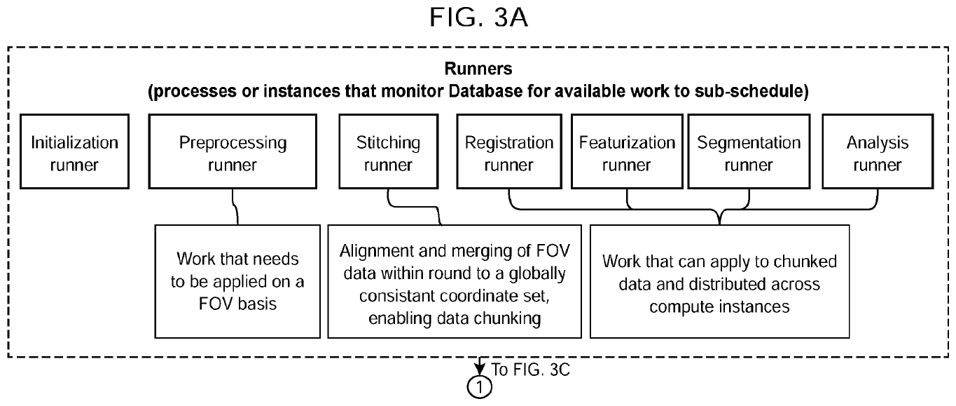

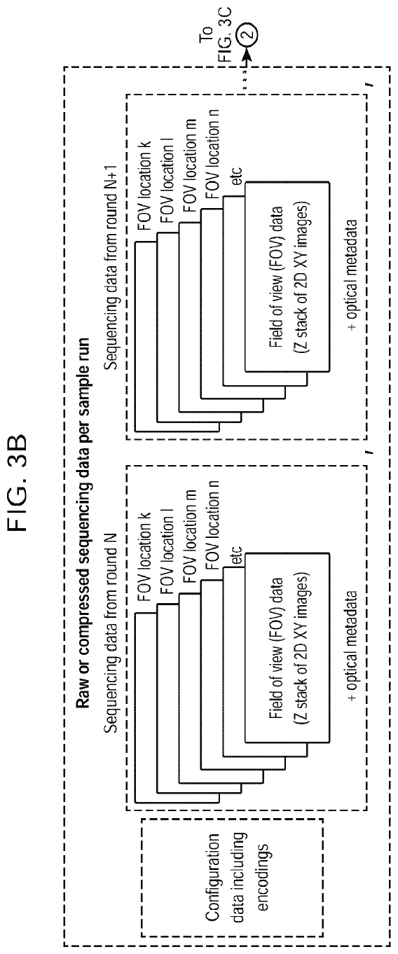

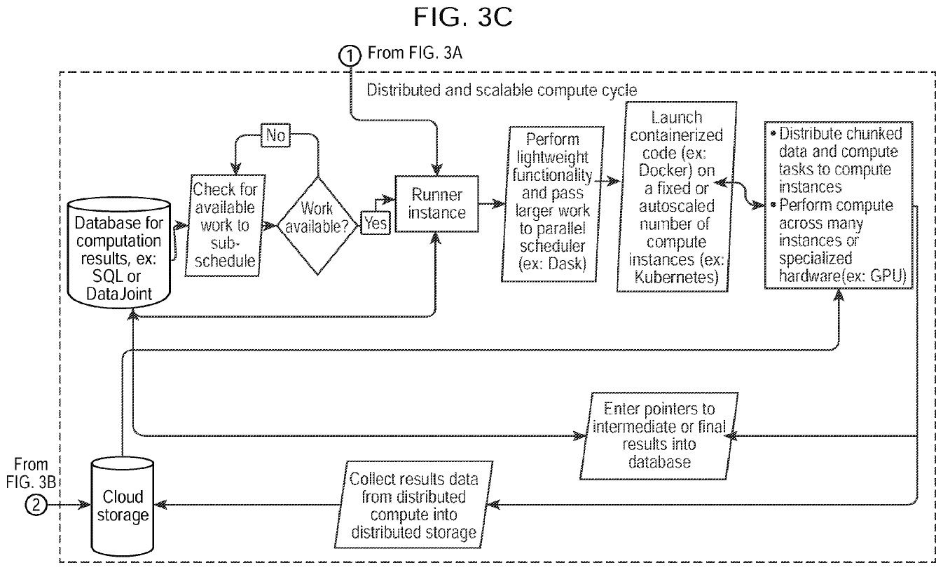

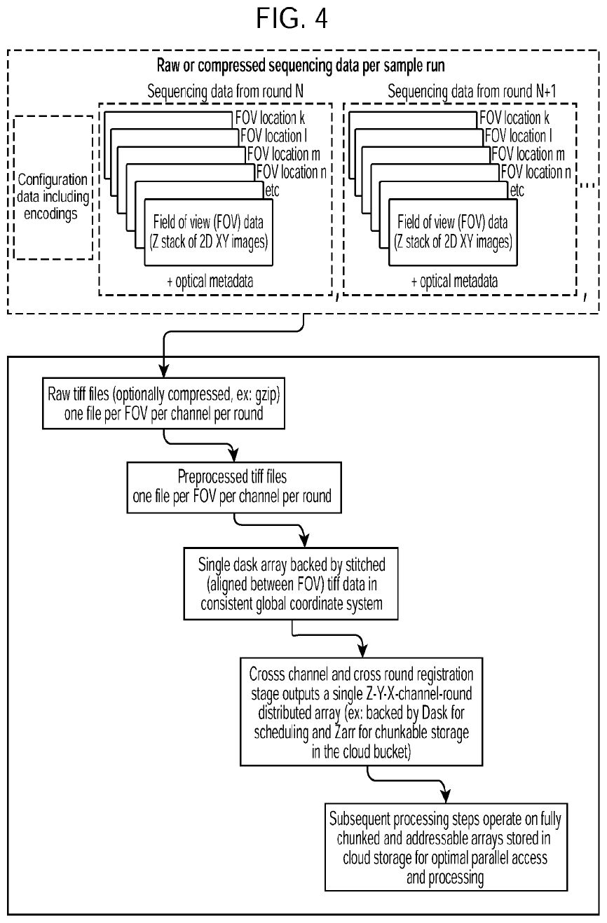

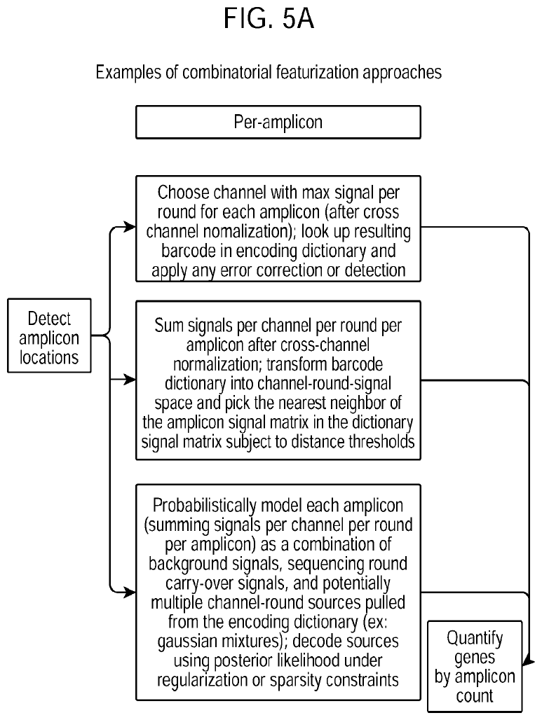

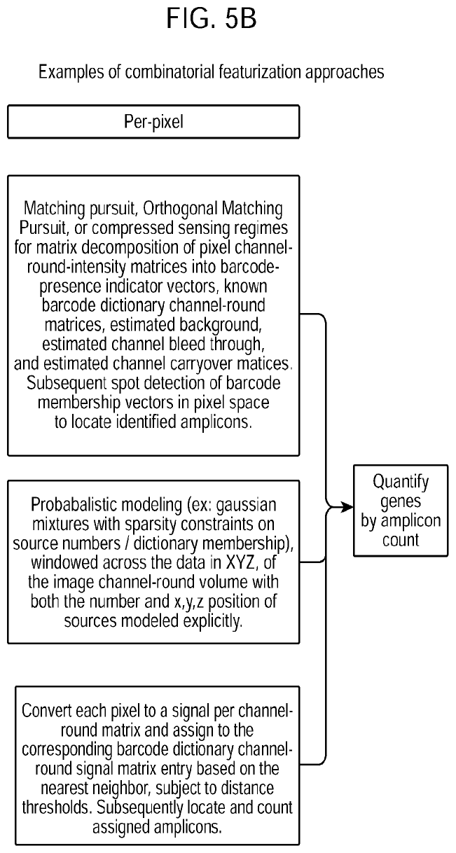

### DETAILED DESCRIPTION OF THE INVENTION

Methods, systems, and devices, including computer programs encoded on a computer storage medium are provided for processing of next-generation in situ sequencing data for nucleic acids in cells in tissue. In particular, cloud-based scalable data processing software for volumetric in situ sequencing is provided.

Before the cloud-based scalable data processing software for volumetric in situ sequencing and computer systems and methods of using it are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the nucleic acid” includes reference to one or more nucleic acids and equivalents thereof, e.g., oligonucleotides or polynucleotides known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “about”, particularly in reference to a given quantity, is meant to encompass deviations of plus or minus five percent.

The terms “peptide”, “oligopeptide”, “polypeptide”, and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymers. Both full-length proteins and fragments thereof are encompassed by the definition. The terms also include post-expression modifications of the polypeptide, for example, phosphorylation, glycosylation, acetylation, hydroxylation, oxidation, and the like as well as chemically or biochemically modified or derivatized amino acids and polypeptides having modified peptide backbones. The terms also include fusion proteins, including, but not limited to, fusion proteins with a heterologous amino acid sequence, fusions with heterologous and homologous leader sequences, with or without N-terminal methionine residues; immunologically tagged proteins; and the like. The terms include polypeptides including one or more of a fatty acid moiety, a lipid moiety, a sugar moiety, and a carbohydrate moiety.

As used herein, the term “target nucleic acid” is any polynucleotide nucleic acid molecule (e.g., DNA molecule; RNA molecule, modified nucleic acid, etc.) present in a single cell. In some embodiments, the target nucleic acid is a coding RNA (e.g., mRNA). In some embodiments, the target nucleic acid is a non-coding RNA (e.g., tRNA, rRNA, microRNA (miRNA), mature miRNA, immature miRNA; etc.). In some embodiments, the target nucleic acid is a splice variant of an RNA molecule (e.g., mRNA, pre-mRNA, etc.) in the context of a cell. A suitable target nucleic acid can therefore be an unspliced RNA (e.g., pre-mRNA, mRNA), a partially spliced RNA, or a fully spliced RNA, etc. Target nucleic acids of interest may be variably expressed, i.e. have a differing abundance, within a cell population, wherein the methods of the invention allow profiling and comparison of the expression levels of nucleic acids, including without limitation RNA transcripts, in individual cells. A target nucleic acid can also be a DNA molecule, e.g. a denatured genomic, viral, plasmid, etc.

The terms “oligonucleotide,” “polynucleotide,” and “nucleic acid molecule”, used interchangeably herein, refer to polymeric forms of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases. The backbone of the polynucleotide can include sugars and phosphate groups (as may typically be found in RNA or DNA), or modified or substituted sugar or phosphate groups. Alternatively, the backbone of the polynucleotide can include a polymer of synthetic subunits such as phosphoramidites, and/or phosphorothioates, and thus can be an oligodeoxynucleoside phosphoramidate or a mixed phosphoramidate-phosphodiester oligomer. Peyrottes et al. (1996) Nucl. Acids Res. 24:1841-1848; Chaturvedi et al. (1996) Nucl. Acids Res. 24:2318-2323. The polynucleotide may include one or more L-nucleosides. A polynucleotide may include modified nucleotides, such as methylated nucleotides and nucleotide analogs, uracyl, other sugars, and linking groups such as fluororibose and thioate, and nucleotide branches. The sequence of nucleotides may be interrupted by non-nucleotide components. A polynucleotide may be modified to include N3′-P5′ (NP) phosphoramidate, morpholino phosphorociamidate (MF), locked nucleic acid (LNA), 2′-O-methoxyethyl (MOE), or 2′-fluoro, arabino-nucleic acid (FANA), which can enhance the resistance of the polynucleotide to nuclease degradation (see, e.g., Faria et al. (2001) Nature Biotechnol. 19:40-44; Toulme (2001) Nature Biotechnol. 19:17-18). A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support. Immunomodulatory nucleic acid molecules can be provided in various formulations, e.g., in association with liposomes, microencapsulated, etc., as described in more detail herein. A polynucleotide used in amplification is generally single-stranded for maximum efficiency in amplification, but may alternatively be double-stranded. If double-stranded, the polynucleotide can first be treated to separate its strands before being used to prepare extension products. This denaturation step is typically affected by heat, but may alternatively be carried out using alkali, followed by neutralization.

The terms “individual”, “subject”, “host”, and “patient”, are used interchangeably herein and refer to invertebrates and vertebrates including, but not limited to, arthropods (e.g., insects, crustaceans, arachnids), cephalopods (e.g., octopuses, squids), amphibians (e.g., frogs, salamanders, caecilians), fish, reptiles (e.g., turtles, crocodilians, snakes, amphisbaenians, lizards, tuatara), mammals, including human and non-human mammals such as non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows; and birds such as domestic, wild and game birds, including chickens, turkeys and other gallinaceous birds, ducks, and geese. In some cases, the methods of the invention find use in experimental animals, in veterinary application, and in the development of animal models for disease, including, but not limited to, rodents including mice, rats, and hamsters; primates, and transgenic animals.

The term “user” as used herein refers to a person that interacts with a device and/or system disclosed herein for performing one or more steps of the presently disclosed methods. The user may be a subject processing in situ sequencing imaging data. In some cases, the subject who processes the in situ sequencing imaging data also performs in situ sequencing to generate the in situ sequencing imaging data.

### Systems and Computer Implemented Methods for Processing In Situ Sequencing Imaging Data

The present disclosure provides systems and computer implemented methods which find use in practicing the subject methods. In some embodiments, the system may include: a processor programmed to process in situ sequencing imaging data, as described herein; and a display component for displaying information regarding the target nucleic acids identified in cells within a tissue sample from in situ sequencing. The system may also comprise one or more graphic boards for processing and outputting graphical information of a tissue image to the display component.

In some embodiments, a computer implemented method is used for processing in situ sequencing imaging data. The processor may be programmed to perform steps of the computer implemented method comprising: (a) receiving in situ sequencing imaging data; (b) applying configuration parameters to the in situ sequencing imaging data, wherein the configuration parameters comprise an encoding scheme, a codebook, image acquisition parameters, and sample metadata; (c) preprocessing imaging data by removing optical aberrations, subtracting background signal, performing deconvolution, filtering the images, and merging overlapping fields of view; (d) performing registration of imaging data by aligning selected common features in images taken at different timepoints and across different color channels at each time point; (e) segmenting images to determine locations of cell nuclei and cells; and (f) performing sequential analysis of images if the in situ sequencing imaging data is from sequential sequencing, wherein said performing sequential analysis of images comprises: i) measuring an intensity of a fluorescent signal for each segmented cell and nucleus, wherein the intensity of the fluorescence signal is proportional to the amount of a target nucleic acid, and ii) subtracting estimated carry-over signal from a previous sequencing round, or performing combinatorial analysis of images if the in situ sequencing imaging data is from combinatorial sequencing, wherein said performing sequential analysis of images comprises i) identifying each amplicon labeling a target nucleic acid from detection of barcodes, wherein invalid barcodes are removed according to their encoding scheme, ii) measuring a fluorescent signal of each amplicon at each timepoint to determine which color channel has the highest signal for each detected amplicon, closest code-book entry to a vector of signals, or clustering of pixel intensities; iii) iteratively matching signals across rounds of sequencing to membership in the code-book; iv) assigning a target identity to each detected amplicon, and v) calculating the number of each target nucleic acid present in each cell and nucleus. In certain embodiments the imaging data comprises morphology images, images from sequential sequencing, or images from combinatorial sequencing, or a combination thereof. In certain embodiments, imaging data is divided into chunks for processing by a plurality of graphics processing units (GPUs).

In certain embodiments, the method further comprises displaying a target identity and target location for each identified target nucleic acid superimposed on a processed image of the tissue. In some embodiments, the method further comprises displaying the number of each target nucleic acid present in each cell and nucleus.

In certain embodiments, the method further comprises optimizing processing parameters by performing multiple rounds of processing of the in situ sequencing imaging data by reiterating steps (a)-(f), wherein different configuration parameters are used to process the in situ sequencing imaging data for each round of data processing.

In certain embodiments, the imaging data comprises images taken at multiple timepoints.

In certain embodiments, the imaging data comprises images from multiple color channels at each time point.

In certain embodiments, the imaging data further comprises morphological information, sequential readout amplicon data, or a single base of combinatorial readout amplicon data.

In certain embodiments, performing registration comprises aligning images based on detection of a common imaging dye used to stain a cell component. In some embodiments, the common imaging dye is a DNA dye used to stain nuclei. In some embodiments, the DNA dyes is a fluorescent DNA dye. An exemplary DNA dye includes, without limitation, 4′,6-diamidino-2-phenylindole (DAPI). In certain embodiments, performing registration comprises aligning images based on detection of immunofluorescence staining of a cell component. In some embodiments, the cell component is a cell membrane marker. In certain embodiments, performing registration comprises aligning images based on detection of immunofluorescence staining of a cell component. In some embodiments, the cell component is a cell membrane marker. In certain embodiments, performing registration comprises aligning images based on detection of fluorescently labeled amplicons. In some embodiments, performing registration comprises aligning images taken at each time point based on detection of a fluorophore used to label amplicons. In some embodiments, the method further comprises aligning images from combinatorial sequencing taken at different times or from different color channels. In certain embodiments, performing registration of imaging data from combinatorial sequencing comprises performing intra-channel registration to generate intra-channel registered data; and performing inter-channel registration on the intra-channel registered data. In some embodiments, overlap in amplicon features is used to align images for intra-channel registration at a sub-pixel level. In some embodiments, inter-channel registration comprises performing a roundmax-projection on the intra-channel registered data.

In certain embodiments, the method further comprises outputting aligned images of all amplicons appearing in a color channel for each channel for each time point.

In certain embodiments, segmenting comprises segmenting images based on detecting cell nuclei. In some embodiments, segmenting further comprises segmenting images based on detecting cells. In certain embodiments, segmenting images further comprises performing distributed stitching segmentation to stitch together cells that cross chunk boundaries if imaging data is divided into chunks for processing by multiple GPUs.

In certain embodiments for sequential sequencing, estimating carry-over signal comprises identifying isolated amplicons for a given color channel and round of sequencing, measuring a fluorescent signal for each isolated amplicon across subsequent time points in all color channels, and selecting the isolated amplicon having the brightest fluorescent signal to measure median carry-over fraction from a given detection time point to subsequent measurement time points. In some embodiments, the method further comprises correcting fluorescent signals for a cell by calculating the true signal (S_true) based on the measured signal (S_measured) and the carry over matrix (M_co) according to the equation: S_measured=S_true*M_co.

In certain embodiments, a computer implemented method is provided, the method comprising: detecting amplicon locations by a method comprising: choosing a channel with the maximum signal per round of sequencing for each amplicon; finding barcodes of the amplicons in an encoding dictionary; applying error correction; performing cross-channel normalization; summing signals per channel per round per amplicon; performing probabilistic modeling of each amplicon based on the summing signals per channel per amplicon, background signals, and carry-over signals per sequencing round; and decoding sources of signals using posterior likelihood probabilities with regularization or sparsity constraints.

In certain embodiments, a computer implemented method is provided, the method comprising: performing a matching pursuit, an orthogonal matching pursuit, or a compressed sensing regime for matrix decomposition of pixel channel-round-intensity matrices into barcode-presence indicator vectors, barcode dictionary channel-round matrices, estimated background, estimated channel bleed through, and estimated channel carryover matrices; identifying a location of an amplicon by detecting barcode membership vectors in pixel space; performing probabilistic modeling of the image channel-round volume with both the number and x, y, and z coordinates of sources; converting each pixel to a signal per a channel-round matrix; assigning the corresponding barcode dictionary channel-round signal matrix entry based on a nearest neighbor; and locating and counting assigned amplicons.

The method can be implemented in digital electronic circuitry, or in computer software, firmware, or hardware. The disclosed and other embodiments can be implemented as one or more computer program products, i.e., one or more modules of computer program instructions encoded on a computer readable medium for execution by, or to control the operation of, a data processing apparatus. The computer readable medium can be a machine-readable storage device, a machine-readable storage substrate, a memory device, a composition of matter effecting a machine-readable propagated signal, or any combination thereof.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

In a further aspect, the system for performing the computer implemented method, as described, may include a processor, a storage component (i.e., memory), a display component, and other components typically present in general purpose computers. In some embodiments, the processor is provided by a computer or handheld device (e.g., a cell phone or tablet). The storage component stores information accessible by the processor, including instructions that may be executed by the processor and data that may be retrieved, manipulated or stored by the processor.

The storage component includes instructions. For example, the storage component includes instructions for processing in situ sequencing imaging data, as described herein. The computer processor is coupled to the storage component and configured to execute the instructions stored in the storage component in order to receive in situ sequencing imaging data and analyze the data according to one or more algorithms, as described herein. The display component displays information regarding the identified target nucleic acids and the spatial locations of the identified target nucleic acids within the tissue sample. In certain embodiments, the display component displays a target identity and target location for each identified target nucleic acid superimposed on a processed image of the tissue. In some embodiments, the display component further displays the number of each target nucleic acid present in each cell and nucleus. In certain embodiments, the display component displays information regarding the sequential analysis of images if the in situ sequencing imaging data is from sequential sequencing, or displays information regarding the combinatorial analysis of images if the in situ sequencing imaging data is from combinatorial sequencing.

The storage component may be of any type capable of storing information accessible by the processor, such as a hard-drive, memory card, ROM, RAM, DVD, CD-ROM, USB Flash drive, write-capable, and read-only memories. The processor may be any well-known processor, such as processors from Intel Corporation. Alternatively, the processor may be a dedicated controller such as an ASIC.

In certain embodiments, the in situ sequencing imaging data are uploaded and stored in a cloud data storage system. In some embodiments, the cloud data storage system is a public cloud storage system. In other embodiments, the cloud data storage system is a private cloud storage system. Cloud data storage may be used to store raw images, intermediate processed files, and final data products. Processing may begin with the upload of a dataset into cloud storage by a data acquisition system. Configuration parameters such as the encoding scheme, codebook, image acquisition parameters, and sample metadata can be input by a user using a data management web interface or generated automatically from a configuration file uploaded into cloud storage along with the sequencing data. Each set of configuration parameters is stored in a cloud database. In some cases, multiple processing runs using different configuration parameters can be applied to a single dataset to optimize processing parameters.

The instructions may be any set of instructions to be executed directly (such as machine code) or indirectly (such as scripts) by the processor. In that regard, the terms “instructions,” “steps” and “programs” may be used interchangeably herein. The instructions may be stored in object code form for direct processing by the processor, or in any other computer language including scripts or collections of independent source code modules that are interpreted on demand or compiled in advance.

Data may be retrieved, stored or modified by the processor in accordance with the instructions. For instance, although the system is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information which is used by a function to calculate the relevant data.

In certain embodiments, the processor and storage component may comprise multiple processors and storage components that may or may not be stored within the same physical housing. For example, some of the instructions and data may be stored on removable CD-ROM and others within a read-only computer chip. Some or all of the instructions and data may be stored in a location physically remote from, yet still accessible by, the processor. Similarly, the processor may comprise a collection of processors which may or may not operate in parallel. In some embodiments, a hardware accelerator is used. In some embodiments, imaging data is divided into chunks for processing by a plurality of graphics processing units (GPUs), field-programmable gate arrays (FPGAs), or tensor processing units (TPUs).

In some embodiments, the method can be performed using a cloud computing system. In these embodiments, the image data files and the programming can be exported to a cloud computer, which runs the program, and returns an output to the user.

Components of systems for carrying out the presently disclosed methods are further described in the examples below.

### Sequencing Chemistries and Encodings for In Situ Gene Sequencing

Sequencing chemistries and encodings for in situ gene sequencing are provided. In one aspect, a method of sequencing by competitive annealing and ligation to determine a sequence of a target nucleic acid is provided, the method comprising performing one or more sequencing cycles, each cycle comprising: (a) contacting the target nucleic acid with a read oligonucleotide and a set of fluorescently labeled decoding probes, wherein the read oligonucleotide comprises a first complementarity region that is complementary to a reading sequence on the target nucleic acid, and wherein each decoding probe comprises a second complementarity region that is complementary to a probe binding site on the target nucleic acid; (b) ligating the read oligonucleotide to one of the decoding probes of the set of fluorescently labeled decoding probes to generate a fluorescent ligation product, wherein the ligation only occurs when the read oligonucleotide and the decoding probe bind to adjacent sequences on the target nucleic acid and both the read oligonucleotide and the decoding probe have sequences that are exactly complementary to the sequence of the target nucleic acid; (c) removing unligated probes; (d) imaging the fluorescent ligation product to detect the fluorescent label of the decoding probe that ligated to the read oligonucleotide, wherein the fluorescent label identifies a nucleotide of the sequence of the target nucleic acid; and (e) removing the fluorescent ligation product from the target nucleic acid by binding a competitor oligonucleotide to the target nucleic acid, wherein the competitor oligonucleotide comprises a third complementarity region comprising a sequence that is complementary to the reading sequence on the target nucleic acid, wherein the fluorescent ligation product dissociates from the target nucleic acid.

In exemplary aspects, the ligation involves each of the read oligonucleotide and a fluorescently labeled decoding probe ligating to form a stable product for imaging only when a perfect match occurs. In certain aspects, the mismatch sensitivity of a ligase enzyme is used to determine the underlying sequence of the target nucleic acid molecule. Inclusion of a polyethylene glycol (PEG) polymer in the sequencing ligation mixture substantially accelerates signal addition onto target nucleic acids. Exemplary PEG polymers have molecular weights ranging from 300 g/mol to 10,000,000 g/mol. In some embodiments, a PEG 6000 polymer is present during ligation of the read oligonucleotide and a fluorescently labeled decoding probe.

In certain embodiments, the set of fluorescently labeled decoding probes comprises: a first probe encoding a guanine, wherein the first probe comprises a first fluorescent label, a second probe encoding an adenine, wherein the second probe comprises a second fluorescent label, a third probe encoding a cytosine, wherein the third probe comprises a third fluorescent label, and a fourth probe encoding a thymine, wherein the fourth probe comprises a fourth fluorescent label.

In certain embodiments, each fluorescently labeled decoding probe encodes 1 to 3 bases adjacent to a ligation junction where the read oligonucleotide is ligated to the fluorescently labeled decoding probe, wherein fluorescently labeled decoding probes encoding different sequences of bases comprise different fluorescent labels.

In certain embodiments, the sequences of the fluorescently labeled decoding probes for a current cycle of sequencing are optimized to minimize cross-hybridization with the fluorescently labeled decoding probes for other sequencing cycles.

In certain embodiments, the read oligonucleotide ranges in length from 8 to 11 nucleotides, including any length within this range such as 8, 9, 10, or 11 nucleotides in length. In some embodiments, the read oligonucleotide has a melting temperature ranging from 17° C. to 20° C., including any melting temperature within this range such as 17° C., 18° C., 19° C., or 20° C.

In certain embodiments, the competitor oligonucleotide further comprises a fourth complementarity region comprising a sequence that is complementary to at least a portion of the probe binding site. In some embodiments, the fourth complementarity region of the competitor oligonucleotide comprises a sequence that is fully complementary to the entire probe binding site on the target nucleic acid. In certain embodiments, the competitor oligonucleotide further comprises a fifth complementarity region comprising a sequence that is complementary to a competitor-specific complementary site adjacent to the reading sequence on the target nucleic acid. In some embodiments, the competitor-specific complementary site ranges in length from 2 nucleotides to 16 nucleotides, including any length within this range such as 2, 3, 4, 5, 6, 7, 8, 9, 10, 11, 12, 13, 14, 15, or 16 nucleotides. In certain embodiments, for sequencing cycles following an initial sequencing cycle, the competitor oligonucleotide used in a previous cycle of sequencing is present during one or more subsequent cycles of sequencing.

In certain embodiments, the read oligonucleotide further comprises a competitor-specific complementary sequence. In some embodiments, the competitor-specific complementary sequence of the read oligonucleotide ranges in length from 2 nucleotides to 16 nucleotides, including any length within this range such as 2, 3, 4, 5, 6, 7, 8, 9, 10, 11, 12, 13, 14, 15, or 16 nucleotides. In certain embodiments, the sequence of the read oligonucleotide for a current cycle of sequencing is optimized to minimize cross-hybridization with read oligonucleotides for other sequencing cycles.

In certain embodiments, multiple read oligonucleotides, sets of fluorescently labeled decoding probes, and competitor oligonucleotides having specificity for different target nucleic acids are used to sequence a plurality of different target nucleic acids simultaneously or sequentially.

In certain embodiments, the competitor oligonucleotides remove ligation products from a previous round of sequencing from different target nucleic acids than target nucleic acids currently undergoing steps (a) or (b) of a sequencing cycle. In certain embodiments, the competitor oligonucleotides remove ligation products from a previous round of sequencing from the same target nucleic acids currently undergoing steps (a) or (b) of a sequencing cycle. In certain embodiments, the competitor oligonucleotide is a round-specific competitor oligonucleotide comprising a fourth complementarity region comprising a sequence that is complementary to the reading sequence for the next cycle of sequencing.

The sequencing reads may be in a 5′ to 3′ forward direction or a 3′ to 5′ reverse direction. For sequencing reads in the forward direction, each fluorescently labeled decoding probe has a fluorophore modification at the 5′ end and each read oligonucleotide has a phosphate at the 5′ end. For sequencing reads in the reverse direction, each fluorescently labeled decoding probe has a phosphate at the 5′ end and a fluorophore modification at the 3′ end.

In certain embodiments, the sequencing is performed with sequential encoding. In some embodiments, each read oligonucleotide comprises a unique sequential orthogonal readout sequence and a unique adjacent competitor-specific complementary sequence for each cycle of sequencing. In some embodiments, the unique sequential orthogonal readout sequence ranges in length from 8 nucleotides to 11 nucleotides, including any length within this range such as 8, 9, 10, or 11 nucleotides. In some embodiments, the unique adjacent competitor-specific complementary sequence ranges in length from 2 nucleotides to 16 nucleotides, including any length within this range such as 2, 3, 4, 5, 6, 7, 8, 9, 10, 11, 12, 13, 14, 15, or 16 nucleotides. In some embodiments, each competitor oligonucleotide comprises a sequence that is complementary to the unique sequential orthogonal readout sequence and the unique adjacent competitor-specific complementary sequence of the read oligonucleotide and at least a portion of the sequence of the fluorescently labeled decoding probe for each cycle of sequencing. In some embodiments, the sequence of the competitor oligonucleotide has partial complementarity or full complementarity to the sequence of the fluorescently labeled decoding probe.

In certain embodiments, sequencing is performed with combinatorial encoding. In some embodiments, multiple read oligonucleotides are used for sequencing, wherein each read oligonucleotide comprises a first complementarity region comprising a combinatorial readout sequence that is complementary to a reading sequence at a separate combinatorial read position on the target nucleic acid, wherein the reading sequence at each separate position on the target nucleic is adjacent to a probe binding site. In some embodiments, each read oligonucleotide further comprises a competitor-specific complementary sequence adjacent to the reading sequence. In some embodiments, the competitor-specific complementary sequence is not complementary to the fluorescently labeled decoding probe. In some embodiments, the reading sequence ranges in length from 8 nucleotides to 11 nucleotides, including any length within this range such as 8, 9, 10, or 11 nucleotides. In some embodiments, the competitor-specific complementary sequence ranges in length from 2 nucleotides to 16 nucleotides, including any length within this range such as 2, 3, 4, 5, 6, 7, 8, 9, 10, 11, 12, 13, 14, 15, or 16 nucleotides. In some embodiments, for each separate combinatorial read position, the competitor oligonucleotide comprises a sequence that is complementary to the combinatorial readout sequence and the competitor-specific complementary sequence of the read oligonucleotide and at least a portion of the sequence of the fluorescently labeled decoding probe for each cycle of sequencing. In some embodiments, the combinatorial encoding uses a hamming code (see Example 2).

The sequencing methods described herein can be used for situ gene sequencing of a target nucleic acid in a cell in an intact tissue. In some embodiments, the method of in situ gene sequencing of a target nucleic acid in a cell in an intact tissue comprises: (a) contacting a fixed and permeabilized intact tissue with at least a pair of oligonucleotide primers under conditions to allow for specific hybridization, wherein the pair of primers comprise a first oligonucleotide and a second oligonucleotide; wherein each of the first oligonucleotide and the second oligonucleotide comprises a first complementarity region, a second complementarity region sequence, and a third complementarity region; wherein the second oligonucleotide further comprises a barcode sequence; wherein the first complementarity region of the first oligonucleotide is complementary to a first portion of the target nucleic acid, wherein the second complementarity region of the first oligonucleotide is complementary to the first complementarity region of the second oligonucleotide, wherein the third complementarity region of the first oligonucleotide is complementary to the third complementarity region of the second oligonucleotide, wherein the second complementary region of the second oligonucleotide is complementary to a second portion of the target nucleic acid, wherein the first portion of the target nucleic is adjacent to the second portion of the target nucleic acid; (b) adding ligase to ligate the second oligonucleotide and generate a closed nucleic acid circle; (c) performing rolling circle amplification in the presence of a nucleic acid molecule, wherein the performing comprises using the second oligonucleotide as a template and the first oligonucleotide as a primer for a polymerase to form one or more amplicons; (d) embedding the one or more amplicons in the presence of hydrogel subunits to form one or more hydrogel-embedded amplicons; (e) sequencing the one or more amplicons according to a method described herein. In certain embodiments, the sequencing is performed with sequential encoding. In other embodiments, the sequencing is performed with combinatorial encoding.

In some embodiments, the contacting the one or more hydrogel-embedded amplicons occurs two times or more, including, but not limited to, e.g., three times or more, four times or more, five times or more, six times or more, or seven times or more. In certain embodiments, the contacting the one or more hydrogel-embedded amplicons occurs four times or more for thin tissue specimens. In other embodiments, the contacting the one or more hydrogel-embedded amplicons occurs six times or more for thick tissue specimens. In some embodiments, one or more amplicons can be contacted by a pair of primers for 24 or more hours, 24 or less hours, 18 or less hours, 12 or less hours, 8 or less hours, 6 or less hours, 4 or less hours, 2 or less hours, 60 or less minutes, 45 or less minutes, 30 or less minutes, 25 or less minutes, 20 or less minutes, 15 or less minutes, 10 or less minutes, 5 or less minutes, or 2 or less minutes. In some embodiments, the methods are performed at room temperature for preservation of tissue morphology with low background noise and error reduction. In some embodiments, the contacting the one or more hydrogel-embedded amplicons includes eliminating error accumulation as sequencing proceeds.

Specimens prepared using the subject methods may be analyzed by any of a number of different types of microscopy, for example, optical microscopy (e.g. bright field, oblique illumination, dark field, phase contrast, differential interference contrast, interference reflection, epifluorescence, confocal, etc., microscopy), laser microscopy, electron microscopy, and scanning probe microscopy. In some aspects, a non-transitory computer readable medium transforms raw images acquired through microscopy of multiple rounds of in situ sequencing first into decoded gene identities and spatial locations and then analyzes the per-cell composition of gene expression.

The term “perfectly matched”, when used in reference to a duplex means that the polynucleotide and/or oligonucleotide strands making up the duplex form a double stranded structure with one another such that every nucleotide in each strand undergoes Watson-Crick base pairing with a nucleotide in the other strand. The term “duplex” includes, but is not limited to, the pairing of nucleoside analogs, such as deoxyinosine, nucleosides with 2-aminopurine bases, peptide nucleic acids (PNAs), and the like, that may be employed. A “mismatch” in a duplex between two oligonucleotides means that a pair of nucleotides in the duplex fails to undergo Watson-Crick bonding.

In some embodiments, the method includes a plurality of read oligonucleotides, including, but not limited to, 5 or more read oligonucleotides, e.g., 8 or more, 10 or more, 12 or more, 15 or more, 18 or more, 20 or more, 25 or more, 30 or more, 35 or more that hybridize to target nucleotide sequences. In some embodiments, a method of the present disclosure includes a plurality of read oligonucleotides, including, but not limited to, 15 or more read oligonucleotides, e.g., 20 or more, 30 or more, 40 or more, 50 or more, 60 or more, 70 or more, and up to 80 different read oligonucleotides that hybridize to 15 or more, e.g., 20 or more, 30 or more, 40 or more, 50 or more, 60 or more, 70 or more, and up to 80 different target nucleotide sequences.

In some embodiments, the methods include a plurality of fluorescently labeled decoding probes, including, but not limited to, 4 or more fluorescently labeled decoding probes, e.g., 8 or more, 10 or more, 12 or more, 16 or more, 18 or more, 20 or more, 25 or more, 30 or more, 35 or more. In some embodiments, a method of the present disclosure includes a plurality of fluorescently labeled decoding probes including, but not limited to, 15 or more fluorescently labeled decoding probes, e.g., 20 or more, 30 or more, 40 or more, 50 or more, 60 or more, 70 or more, and up to 80 different fluorescently labeled decoding probes that hybridize to 15 or more, e.g., 20 or more, 30 or more, 40 or more, 50 or more, 60 or more, 70 or more, and up to 80 different target nucleotide sequences.

A plurality of pairs of oligonucleotide primers can be used in a reaction, where one or more pairs specifically bind to each target nucleic acid. For example, two primer pairs can be used for one target nucleic acid in order to improve sensitivity and reduce variability. It is also of interest to detect a plurality of different target nucleic acids in a cell, e.g. detecting up to 2, up to 3, up to 4, up to 5, up to 6, up to 7, up to 8, up to 9, up to 10, up to 12, up to 15, up to 18, up to 20, up to 25, up to 30, up to 40 or more distinct target nucleic acids.

In certain embodiments, sequencing is performed with a ligase with activity hindered by base mismatches, a read oligonucleotide, and a fluorescently labeled decoding probe. The term “hindered” in this context refers to activity of a ligase that is reduced by approximately 20% or more, such as by 25% or more, such as by 50% or more, such as by 75% or more, such as by 90% or more, such as by 95% or more, such as by 99% or more, such as by 100%. In some embodiments, the third oligonucleotide has a length of 5-15 nucleotides, including, but not limited to, 5-13 nucleotides, 5-10 nucleotides, or 5-8 nucleotides. In some embodiments, the Tₘ of the third oligonucleotide is at room temperature (22-25° C.). In some embodiments, the read oligonucleotide is degenerate, or partially thereof. In some embodiments, the fluorescently labeled decoding probe oligonucleotide has a length of 5-15 nucleotides, including, but not limited to, 5-13 nucleotides, 5-10 nucleotides, or 5-8 nucleotides. In some embodiments, the Tₘ of the fourth oligonucleotide is at room temperature (22°-25° C.). After each cycle of sequencing corresponding to a base readout, the fluorescent ligation product is removed from the target nucleic acid by binding a competitor oligonucleotide to the target nucleic acid, wherein the competitor oligonucleotide comprises a third complementarity region comprising a sequence that is complementary to the reading sequence on the target nucleic acid, wherein the fluorescent ligation product dissociates from the target nucleic acid.

In some embodiments, sequencing involves washing to remove unbound oligonucleotides and unligated probes, thereafter revealing a fluorescent product for imaging. In certain exemplary embodiments, a detectable fluorescent label is used to detect one or more nucleotides and/or oligonucleotides described herein. In certain embodiments, a detectable fluorescent label such as a fluorescent protein, fluorescent dye, or fluorescent quantum dot is used to label probes.

Fluorescent labels and their attachment to nucleotides and/or oligonucleotides are described in many reviews, including Haugland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). Particular methodologies applicable to the invention are disclosed in the following sample of references: U.S. Pat. Nos. 4,757,141, 5,151,507 and 5,091,519. In one aspect, one or more fluorescent dyes are used as labels for labeled target sequences, e.g., as disclosed by U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); Lee et al.; U.S. Pat. No. 5,066,580 (xanthine dyes); U.S. Pat. No. 5,688,648 (energy transfer dyes); and the like. Labelling can also be carried out with quantum dots, as disclosed in the following patents and patent publications: U.S. Pat. Nos. 6,322,901, 6,576,291, 6,423,551, 6,251,303, 6,319,426, 6,426,513, 6,444,143, 5,990,479, 6,207,392, 2002/0045045 and 2003/0017264. As used herein, the term “fluorescent label” includes a signaling moiety that conveys information through the fluorescent absorption and/or emission properties of one or more molecules. Such fluorescent properties include fluorescence intensity, fluorescence lifetime, emission spectrum characteristics, energy transfer, and the like.

Commercially available fluorescent nucleotide analogues readily incorporated into nucleotide and/or oligonucleotide sequences include, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHODAMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHODAMINE GREEN™-5-UTP, ALEXA FLUOR™ 488-5-UTP, LEXA FLUOR™ 546-14-UTP (Molecular Probes, Inc. Eugene, Oreg.) and the like. Protocols are known in the art for custom synthesis of nucleotides having other fluorophores (See, Henegariu et al. (2000) Nature Biotechnol. 18:345).

Other fluorophores available for post-synthetic attachment include, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, Cy7 (Amersham Biosciences, Piscataway, N.J.) and the like. FRET tandem fluorophores may also be used, including, but not limited to, PerCP-Cy5.5, PE-Cy5, PE-Cy5.5, PE-Cy7, PE-Texas Red, APC-Cy7, PE-Alexa dyes (610, 647, 680), APC-Alexa dyes and the like.

Examples of fluorescent proteins include, but are not limited to, green fluorescent protein, superfolder green fluorescent protein, enhanced green fluorescent protein, Dronpa (a photoswitchable green fluorescent protein), yellow-green fluorescent protein, yellow fluorescent protein, red fluorescent protein, orange fluorescent protein, blue fluorescent protein, cyan fluorescent protein, violet fluorescent protein, mApple, mNectarine, mNeptune, mCherry, mStrawberry, mPlum, mRaspberry, mCrimson3, mCarmine, mCardinal, mScarlet, mRuby2, FusionRed, mNeonGreen, TagRFP675, and mRFP1. and the like.

Metallic silver or gold particles may be used to enhance signal from fluorescently labeled nucleotide and/or oligonucleotide sequences (Lakowicz et al. (2003) Bio Techniques 34:62).

Biotin, or a derivative thereof, may also be used as a label on a nucleotide and/or an oligonucleotide sequence, and subsequently bound by a detectably labeled avidin/streptavidin derivative (e.g. phycoerythrin-conjugated streptavidin), or a detectably labeled anti-biotin antibody. Digoxigenin may be incorporated as a label and subsequently bound by a detectably labeled anti-digoxigenin antibody (e.g. fluoresceinated anti-digoxigenin). An aminoallyl-dUTP residue may be incorporated into an oligonucleotide sequence and subsequently coupled to an N-hydroxy succinimide (NHS) derivatized fluorescent dye. In general, any member of a conjugate pair may be incorporated into a detection oligonucleotide provided that a detectably labeled conjugate partner can be bound to permit detection. As used herein, the term antibody refers to an antibody molecule of any class, or any sub-fragment thereof, such as an Fab.

Other suitable labels for an oligonucleotide sequence may include fluorescein (FAM), digoxigenin, dinitrophenol (DNP), dansyl, biotin, bromodeoxyuridine (BrdU), hexahistidine (6×His), phosphor-amino acids (e.g. P-tyr, P-ser, P-thr) and the like. In one embodiment the following hapten/antibody pairs are used for detection, in which each of the antibodies is derivatized with a detectable label: biotin/α-biotin, digoxigenin/α-digoxigenin, dinitrophenol (DNP)/α-DNP, 5-Carboxyfluorescein (FAM)/α-FAM.

In certain exemplary embodiments, a nucleotide and/or an oligonucleotide sequence can be indirectly labeled, especially with a hapten that is then bound by a capture agent, e.g., as disclosed in U.S. Pat. Nos. 5,344,757, 5,702,888, 5,354,657, 5,198,537 and 4,849,336, PCT publication WO 91/17160 and the like. Many different hapten-capture agent pairs are available for use. Exemplary haptens include, but are not limited to, biotin, des-biotin and other derivatives, dinitrophenol, dansyl, fluorescein, CY5, digoxigenin and the like. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some embodiments, an antioxidant compound is included in the washing and imaging buffers (i.e., “anti-fade buffers”) to reduce photobleaching during fluorescence imaging. Exemplary antioxidants include, without limitation, Trolox (6-hydroxy-2,5,7,8-tetramethylchroman-2-carboxylic acid) and Trolox-quinone, propyl-gallate, tertiary butylhydroquinone, butylated hydroxyanisole, butylated hydroxytoluene, glutathione, ascorbic acid, and tocopherols. Such antioxidants have an antifade effect on fluorophores. That is, the antioxidant reduces photobleaching during tiling, greatly enhances the signal-to-noise ratio (SNR) of sensitive fluorophores, and enables higher SNR imaging of thicker samples. For a fixed exposure time, including an antioxidant increases the SNR by increasing the concentration of the non-bleached fluorophore during exposure to light. Including an antioxidant also removes the diminishing returns of longer exposure times (caused by the limited fluorophore lifetime before photobleaching), providing for increased SNR by allowing increased exposure times.

An exemplary sequencing cycle optionally begins with a brief sample wash, before proceeding to the first signal addition. Depending on whether sequential or combinatorial encoding is being used for a particular round, the corresponding set of read oligonucleotides, fluorescently labeled decoding probes, and their round-specific competitors are added and ligated. In combinatorial encodings, the read oligonucleotide for a given position x is added, plus a set of fluorescently labeled dibase-encoding oligonucleotides, plus a competitor oligonucleotide for the previous position that was labeled (unless it is the first round of labeling, in which case competitor oligonucleotide is omitted). In sequential encodings, the read oligonucleotide for a given round x, a 4-channel fluorophore mixture, and a round x-1 competitor oligonucleotide are added, except if it is the first round of labeling. The presence of PEG in the sequencing ligation mixture substantially accelerates the signal addition onto the target. Following incubation of the sample in imaging buffer, the sample is imaged, and briefly rinsed before proceeding to the next sequencing cycle.

The methods disclosed herein also provide for a method of screening a candidate agent to determine whether the candidate agent modulates gene expression of a nucleic acid in a cell in an intact tissue by performing a method described herein to determine the gene sequence of a target nucleic acid in the cell in the intact tissue, and detecting the level of gene expression of the target nucleic acid, wherein an alteration in the level of expression of the target nucleic acid in the presence of the candidate agent relative to the level of expression of the target nucleic acid in the absence of the candidate agent indicates that the candidate agent modulates gene expression of the nucleic acid in the cell in the intact tissue.

In certain aspects, the methods disclosed herein provide for a faster processing time, higher multiplexity (up to 1000 genes), higher efficiency, higher sensitivity, lower error rate, and more spatially resolved cell types, as compared to existing gene expression analysis tools. The methods provide improved sequencing-by-ligation techniques (SCAL and SEDAL2) for in situ sequencing with error reduction. In some other aspects, the methods disclosed herein include spatially sequencing (e.g. reagents, chips or services) for biomedical research and clinical diagnostics (e.g. cancer, bacterial infection, viral infection, etc.) with single-cell and/or single-molecule sensitivity.

### Specific Amplification of Nucleic Acids Via Intramolecular Ligation (SNAIL)

An efficient approach for generating cDNA libraries from cellular RNAs in situ may be utilized, which is referred to herein as SNAIL, for Specific Amplification of Nucleic Acids via Intramolecular Ligation. In certain embodiments, the method includes contacting a fixed and permeabilized intact tissue with at least a pair of oligonucleotide primers under conditions to allow for specific hybridization, wherein the pair of primers includes a first oligonucleotide and a second oligonucleotide.

More generally, the nucleic acid present in a cell of interest in a tissue serves as a scaffold for an assembly of a complex that includes a pair of primers, referred to herein as a first oligonucleotide and a second oligonucleotide. In some embodiments, the contacting the fixed and permeabilized intact tissue includes hybridizing the pair of primers to the same target nucleic acid. In some embodiments, the target nucleic acid is RNA. In such embodiments, the target nucleic acid may be mRNA. In other embodiments, the target nucleic acid is DNA.

As used herein, the terms “hybridize” and “hybridization” refer to the formation of complexes between nucleotide sequences which are sufficiently complementary to form complexes via Watson-Crick base pairing. Where a primer “hybridizes” with target (template), such complexes (or hybrids) are sufficiently stable to serve the priming function required by, e.g., the DNA polymerase to initiate DNA synthesis. It will be appreciated that the hybridizing sequences need not have perfect complementarity to provide stable hybrids. In many situations, stable hybrids will form where fewer than about 10% of the bases are mismatches, ignoring loops of four or more nucleotides. Accordingly, as used herein the term “complementary” refers to an oligonucleotide that forms a stable duplex with its “complement” under assay conditions, generally where there is about 90% or greater homology.

### SNAIL Oligonucleotide Primers

In the subject methods, the SNAIL oligonucleotide primers include at least a first oligonucleotide and a second oligonucleotide; wherein each of the first oligonucleotide and the second oligonucleotide includes a first complementarity region, a second complementarity region, and a third complementarity region; wherein the second oligonucleotide further includes a barcode sequence; wherein the first complementarity region of the first oligonucleotide is complementary to a first portion of the target nucleic acid, wherein the second complementarity region of the first oligonucleotide is complementary to the first complementarity region of the second oligonucleotide, wherein the third complementarity region of the first oligonucleotide is complementary to the third complementarity region of the second oligonucleotide, wherein the second complementary region of the second oligonucleotide is complementary to a second portion of the target nucleic acid, and wherein the first complementarity region of the first oligonucleotide is adjacent to the second complementarity region of the second oligonucleotide. In an alternative embodiment, the second oligonucleotide is a closed circular molecule, and a ligation step is omitted.

The present disclosure provides methods where the contacting a fixed and permeabilized tissue includes hybridizing a plurality of oligonucleotide primers having specificity for different target nucleic acids. In some embodiments, the methods include a plurality of first oligonucleotides, including, but not limited to, 5 or more first oligonucleotides, e.g., 8 or more, 10 or more, 12 or more, 15 or more, 18 or more, 20 or more, 25 or more, 30 or more, 35 or more that hybridize to target nucleotide sequences. In some embodiments, a method of the present disclosure includes a plurality of first oligonucleotides, including, but not limited to, 15 or more first oligonucleotides, e.g., 20 or more, 30 or more, 40 or more, 50 or more, 60 or more, 70 or more, and up to 80 different first oligonucleotides that hybridize to 15 or more, e.g., 20 or more, 30 or more, 40 or more, 50 or more, 60 or more, 70 or more, and up to 80 different target nucleotide sequences. In some embodiments, the methods include a plurality of second oligonucleotides, including, but not limited to, 5 or more second oligonucleotides, e.g., 8 or more, 10 or more, 12 or more, 15 or more, 18 or more, 20 or more, 25 or more, 30 or more, 35 or more. In some embodiments, a method of the present disclosure includes a plurality of second oligonucleotides including, but not limited to, 15 or more second oligonucleotides, e.g., 20 or more, 30 or more, 40 or more, 50 or more, 60 or more, 70 or more, and up to 80 different first oligonucleotides that hybridize to 15 or more, e.g., 20 or more, 30 or more, 40 or more, 50 or more, 60 or more, 70 or more, and up to 80 different target nucleotide sequences. A plurality of oligonucleotide pairs can be used in a reaction, where one or more pairs specifically bind to each target nucleic acid. For example, two primer pairs can be used for one target nucleic acid in order to improve sensitivity and reduce variability. It is also of interest to detect a plurality of different target nucleic acids in a cell, e.g. detecting up to 2, up to 3, up to 4, up to 5, up to 6, up to 7, up to 8, up to 9, up to 10, up to 12, up to 15, up to 18, up to 20, up to 25, up to 30, up to 40 or more distinct target nucleic acids. The primers are typically denatured prior to use, typically by heating to a temperature of at least about 50° C., at least about 60° C., at least about 70° C., at least about 80° C., and up to about 99° C., up to about 95° C., up to about 90° C.

In some embodiments, the primers are denatured by heating before contacting the sample. In certain aspects, the melting temperature (Tₘ) of oligonucleotides is selected to minimize ligation in solution. The “melting temperature” or “Tm” of a nucleic acid is defined as the temperature at which half of the helical structure of the nucleic acid is lost due to heating or other dissociation of the hydrogen bonding between base pairs, for example, by acid or alkali treatment, or the like. The Tₘ of a nucleic acid molecule depends on its length and on its base composition. Nucleic acid molecules rich in GC base pairs have a higher Tₘ than those having an abundance of AT base pairs. Separated complementary strands of nucleic acid spontaneously reassociate or anneal to form duplex nucleic acid when the temperature is lowered below the Tₘ. The highest rate of nucleic acid hybridization occurs approximately 25 degrees C. below the Tₘ. The Tₘ may be estimated using the following relationship: Tₘ=69.3+0.41(GC) % (Marmur et al. (1962) J. Mol. Biol. 5:109-118).

In certain embodiments, the plurality of second oligonucleotides includes a padlock probe. In some embodiments, the probe includes a detectable label that can be measured and quantitated. The terms “label” and “detectable label” refer to a molecule capable of detection, including, but not limited to, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. The term “fluorescer” refers to a substance or a portion thereof that is capable of exhibiting fluorescence in the detectable range. Particular examples of labels that may be used with the invention include, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenicol acetyl transferase, and urease.

In some embodiments, the one or more first oligonucleotides and second oligonucleotides bind to a different region of the target nucleic acid, or target site. In a pair, each target site is different, and the target sites are adjacent sites on the target nucleic acid, e.g. usually not more than 15 nucleotides distant, e.g. not more than 10, 8, 6, 4, or 2 nucleotides distant from the other site, and may be contiguous sites. Target sites are typically present on the same strand of the target nucleic acid in the same orientation. Target sites are also selected to provide a unique binding site, relative to other nucleic acids present in the cell. Each target site is generally from about 19 to about 25 nucleotides in length, e.g. from about 19 to 23 nucleotides, from about 19 to 21 nucleotides, or from about 19 to 20 nucleotides. The pair of first and second oligonucleotides are selected such that each oligonucleotide in the pair has a similar melting temperature for binding to its cognate target site, e.g. the Tₘ may be from about 50° C., from about 52° C., from about 55° C., from about 58°, from about 62° C., from about 65° C., from about 70° C., or from about 72° C. The GC content of the target site is generally selected to be no more than about 20%, no more than about 30%, no more than about 40%, no more than about 50%, no more than about 60%, no more than about 70%,

In some embodiments, the first oligonucleotide includes a first, second, and third complementarity region. The target site of the first oligonucleotide may refer to the first complementarity region. As summarized above, the first complementarity region of the first oligonucleotide may have a length of 19-25 nucleotides. In certain aspects, the second complementarity region of the first oligonucleotide has a length of 3-10 nucleotides, including, e.g., 4-8 nucleotides or 4-7 nucleotides. In some aspects, the second complementarity region of the first oligonucleotide has a length of 6 nucleotides. In some embodiments, the third complementarity region of the first oligonucleotide likewise has a length of 6 nucleotides. In such embodiments, the third complementarity region of the first oligonucleotide has a length of 3-10 nucleotides, including, e.g., 4-8 nucleotides or 4-7 nucleotides.

In some embodiments, second first oligonucleotide includes a first, second, and third complementarity region. The target site of the second oligonucleotide may refer to the second complementarity region. As summarized above, the second complementarity region of the second oligonucleotide may have a length of 19-25 nucleotides. In certain aspects, the first complementarity region of the first oligonucleotide has a length of 3-10 nucleotides, including, e.g., 4-8 nucleotides or 4-7 nucleotides. In some aspects, the first complementarity region of the first oligonucleotide has a length of 6 nucleotides. In some aspects, the first complementarity region of the second oligonucleotide includes the 5′ end of the second oligonucleotide. In some embodiments, the third complementarity region of the second oligonucleotide likewise has a length of 6 nucleotides. In such embodiments, the third complementarity region of the second oligonucleotide has a length of 3-10 nucleotides, including, e.g., 4-8 nucleotides or 4-7 nucleotides. In further embodiments, the third complementarity region of the second oligonucleotide includes the 3′ end of the second oligonucleotide. In some embodiments, the first complementarity region of the second oligonucleotide is adjacent to the third complementarity region of the second oligonucleotide.

In some aspects, the second oligonucleotide includes a barcode sequence, wherein the barcode sequence of the second oligonucleotide provides barcoding information for identification of the target nucleic acid. The term “barcode” refers to a nucleic acid sequence that is used to identify a single cell or a subpopulation of cells. Barcode sequences can be linked to a target nucleic acid of interest during amplification and used to trace back the amplicon to the cell from which the target nucleic acid originated. A barcode sequence can be added to a target nucleic acid of interest during amplification by carrying out amplification with an oligonucleotide that contains a region including the barcode sequence and a region that is complementary to the target nucleic acid such that the barcode sequence is incorporated into the final amplified target nucleic acid product (i.e., amplicon).

### Tissue

As described herein, the methods disclosed include in situ sequencing technology of an intact tissue by at least contacting a fixed and permeabilized intact tissue with at least a pair of oligonucleotide primers under conditions to allow for specific hybridization. Tissue specimens suitable for use with the methods described herein generally include any type of tissue specimens collected from living or dead subjects, such as, e.g., biopsy specimens and autopsy specimens, of which include, but are not limited to, epithelium, muscle, connective, and nervous tissue. Tissue specimens may be collected and processed using the methods described herein and subjected to microscopic analysis immediately following processing, or may be preserved and subjected to microscopic analysis at a future time, e.g., after storage for an extended period of time. In some embodiments, the methods described herein may be used to preserve tissue specimens in a stable, accessible and fully intact form for future analysis. In some embodiments, the methods described herein may be used to analyze a previously-preserved or stored tissue specimen. In some embodiments, the intact tissue includes brain tissue such as visual cortex slices. In some embodiments, the intact tissue is a thin slice with a thickness of 5-20 μm, including, but not limited to, e.g., 5-18 μm, 5-15 μm, or 5-10 μm. In other embodiments, the intact tissue is a thick slice with a thickness of 50-200 μm, including, but not limited to, e.g., 50-150 μm, 50-100 μm, or 50-80 μm.

Aspects of the invention include fixing intact tissue. The term “fixing” or “fixation” as used herein is the process of preserving biological material (e.g., tissues, cells, organelles, molecules, etc.) from decay and/or degradation. Fixation may be accomplished using any convenient protocol. Fixation can include contacting the sample with a fixation reagent (i.e., a reagent that contains at least one fixative). Samples can be contacted by a fixation reagent for a wide range of times, which can depend on the temperature, the nature of the sample, and on the fixative(s). For example, a sample can be contacted by a fixation reagent for 24 or less hours, 18 or less hours, 12 or less hours, 8 or less hours, 6 or less hours, 4 or less hours, 2 or less hours, 60 or less minutes, 45 or less minutes, 30 or less minutes, 25 or less minutes, 20 or less minutes, 15 or less minutes, 10 or less minutes, 5 or less minutes, or 2 or less minutes.

A sample can be contacted by a fixation reagent for a period of time in a range of from 5 minutes to 24 hours, e.g., from 10 minutes to 20 hours, from 10 minutes to 18 hours, from 10 minutes to 12 hours, from 10 minutes to 8 hours, from 10 minutes to 6 hours, from 10 minutes to 4 hours, from 10 minutes to 2 hours, from 15 minutes to 20 hours, from 15 minutes to 18 hours, from 15 minutes to 12 hours, from 15 minutes to 8 hours, from 15 minutes to 6 hours, from 15 minutes to 4 hours, from 15 minutes to 2 hours, from 15 minutes to 1.5 hours, from 15 minutes to 1 hour, from 10 minutes to 30 minutes, from 15 minutes to 30 minutes, from 30 minutes to 2 hours, from 45 minutes to 1.5 hours, or from 55 minutes to 70 minutes.

A sample can be contacted by a fixation reagent at various temperatures, depending on the protocol and the reagent used. For example, in some instances a sample can be contacted by a fixation reagent at a temperature ranging from −22° C. to 55° C., where specific ranges of interest include, but are not limited to 50 to 54° C., 40 to 44° C., 35 to 39° C., 28 to 32° C., 20 to 26° C., 0 to 6° C., and −18 to −22° C. In some instances a sample can be contacted by a fixation reagent at a temperature of −20° C., 4° C., room temperature (22-25° C.), 30° C., 37° C., 42° C., or 52° C.

Any convenient fixation reagent can be used. Common fixation reagents include crosslinking fixatives, precipitating fixatives, oxidizing fixatives, mercurials, and the like. Crosslinking fixatives chemically join two or more molecules by a covalent bond and a wide range of cross-linking reagents can be used. Examples of suitable cross-liking fixatives include but are not limited to aldehydes (e.g., formaldehyde, also commonly referred to as “paraformaldehyde” and “formalin”; glutaraldehyde; etc.), imidoesters, NHS (N-Hydroxysuccinimide) esters, and the like. Examples of suitable precipitating fixatives include but are not limited to alcohols (e.g., methanol, ethanol, etc.), acetone, acetic acid, etc. In some embodiments, the fixative is formaldehyde (i.e., paraformaldehyde or formalin). A suitable final concentration of formaldehyde in a fixation reagent is 0.1 to 10%, 1-8%, 1-4%, 1-2%, 3-5%, or 3.5-4.5%, including about 1.6% for 10 minutes. In some embodiments the sample is fixed in a final concentration of 4% formaldehyde (as diluted from a more concentrated stock solution, e.g., 38%, 37%, 36%, 20%, 18%, 16%, 14%, 10%, 8%, 6%, etc.). In some embodiments the sample is fixed in a final concentration of 10% formaldehyde. In some embodiments the sample is fixed in a final concentration of 1% formaldehyde. In some embodiments, the fixative is glutaraldehyde. A suitable concentration of glutaraldehyde in a fixation reagent is 0.1 to 1%. A fixation reagent can contain more than one fixative in any combination. For example, in some embodiments the sample is contacted with a fixation reagent containing both formaldehyde and glutaraldehyde.

The terms “permeabilization” or “permeabilize” as used herein refer to the process of rendering the cells (cell membranes etc.) of a sample permeable to experimental reagents such as nucleic acid probes, antibodies, chemical substrates, etc. Any convenient method and/or reagent for permeabilization can be used. Suitable permeabilization reagents include detergents (e.g., Saponin, Triton X-100, Tween-20, etc.), organic fixatives (e.g., acetone, methanol, ethanol, etc.), enzymes, etc. Detergents can be used at a range of concentrations. For example, 0.001%-1% detergent, 0.05%-0.5% detergent, or 0.1%-0.3% detergent can be used for permeabilization (e.g., 0.1% Saponin, 0.2% tween-20, 0.1-0.3% triton X-100, etc.). In some embodiments methanol on ice for at least 10 minutes is used to permeabilize.

In some embodiments, the same solution can be used as the fixation reagent and the permeabilization reagent. For example, in some embodiments, the fixation reagent contains 0.1%-10% formaldehyde and 0.001%-1% saponin. In some embodiments, the fixation reagent contains 1% formaldehyde and 0.3% saponin.

A sample can be contacted by a permeabilization reagent for a wide range of times, which can depend on the temperature, the nature of the sample, and on the permeabilization reagent(s). For example, a sample can be contacted by a permeabilization reagent for 24 or more hours, 24 or less hours, 18 or less hours, 12 or less hours, 8 or less hours, 6 or less hours, 4 or less hours, 2 or less hours, 60 or less minutes, 45 or less minutes, 30 or less minutes, 25 or less minutes, 20 or less minutes, 15 or less minutes, 10 or less minutes, 5 or less minutes, or 2 or less minutes. A sample can be contacted by a permeabilization reagent at various temperatures, depending on the protocol and the reagent used. For example, in some instances a sample can be contacted by a permeabilization reagent at a temperature ranging from −82° C. to 55° C., where specific ranges of interest include, but are not limited to: 50 to 54° C., 40 to 44° C., 35 to 39° C., 28 to 32° C., 20 to 26° C., 0 to 6° C., −18 to −22° C., and −78 to −82° C. In some instances a sample can be contacted by a permeabilization reagent at a temperature of −80° C., −20° C., 4° C., room temperature (22-25° C.), 30° C., 37° C., 42° C., or 52° C.

In some embodiments, a sample is contacted with an enzymatic permeabilization reagent. Enzymatic permeabilization reagents that permeabilize a sample by partially degrading extracellular matrix or surface proteins that hinder the permeation of the sample by assay reagents. Contact with an enzymatic permeabilization reagent can take place at any point after fixation and prior to target detection. In some instances the enzymatic permeabilization reagent is proteinase K, a commercially available enzyme. In such cases, the sample is contacted with proteinase K prior to contact with a post-fixation reagent. Proteinase K treatment (i.e., contact by proteinase K; also commonly referred to as “proteinase K digestion”) can be performed over a range of times at a range of temperatures, over a range of enzyme concentrations that are empirically determined for each cell type or tissue type under investigation. For example, a sample can be contacted by proteinase K for 30 or less minutes, 25 or less minutes, 20 or less minutes, 15 or less minutes, 10 or less minutes, 5 or less minutes, or 2 or less minutes. A sample can be contacted by 1 μg/ml or less, 2 μg/m or less, 4 μg/ml or less, 8 μg/ml or less, 10 μg/ml or less, 20 μg/ml or less, 30 μg/ml or less, 50 μg/ml or less, or 100 μg/ml or less proteinase K. A sample can be contacted by proteinase K at a temperature ranging from 2° C. to 55° C., where specific ranges of interest include, but are not limited to: 50 to 54° C., 40 to 44° C., 35 to 39° C., 28 to 32° C., 20 to 26° C., and 0 to 6° C. In some instances a sample can be contacted by proteinase K at a temperature of 4° C., room temperature (22-25° C.), 30° C., 37° C., 42° C., or 52° C. In some embodiments, a sample is not contacted with an enzymatic permeabilization reagent. In some embodiments, a sample is not contacted with proteinase K. Contact of an intact tissue with at least a fixation reagent and a permeabilization reagent results in the production of a fixed and permeabilized tissue.

### Ligase

In some embodiments, the methods disclosed include adding ligase to ligate the second oligonucleotide and generate a closed nucleic acid circle. In some embodiments, the adding ligase includes adding DNA ligase. In alternative embodiments, the second oligonucleotide is provided as a closed nucleic acid circle, and the step of adding ligase is omitted. In certain embodiments, ligase is an enzyme that facilitates the sequencing of a target nucleic acid molecule.

The term “ligase” as used herein refers to an enzyme that is commonly used to join polynucleotides together or to join the ends of a single polynucleotide. Ligases include ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases include bacterial ligases such as E. coli DNA ligase and Taq DNA ligase, Ampligase® thermostable DNA ligase (Epicentre® Technologies Corp., part of Illumina®, Madison, Wis.) and phage ligases such as T3 DNA ligase, T4 DNA ligase and T7 DNA ligase and mutants thereof.

### Rolling Circle Amplification

In some embodiments, the methods of the invention include the step of performing rolling circle amplification in the presence of a nucleic acid molecule, wherein the performing includes using the second oligonucleotide as a template and the first oligonucleotide as a primer for a polymerase to form one or more amplicons. In such embodiments, a single-stranded, circular polynucleotide template is formed by ligation of the second nucleotide, which circular polynucleotide includes a region that is complementary to the first oligonucleotide. Upon addition of a DNA polymerase in the presence of appropriate dNTP precursors and other cofactors, the first oligonucleotide is elongated by replication of multiple copies of the template. This amplification product can be readily detected by binding to a detection probe. In some embodiments, the polymerase is preincubated without dNTPs to allow the polymerase to penetrate the sample uniformly before performing rolling circle amplification.

In some embodiments, only when a first oligonucleotide and second oligonucleotide hybridize to the same target nucleic acid molecule, the second oligonucleotide can be circularized and rolling-circle amplified to generate a cDNA nanoball (i.e., amplicon) containing multiple copies of the cDNA. The term “amplicon” refers to the amplified nucleic acid product of a PCR reaction or other nucleic acid amplification process. In some embodiments, amine-modified nucleotides are spiked into the rolling circle amplification reaction.

Techniques for rolling circle amplification are known in the art (see, e.g., Baner et al, Nucleic Acids Research, 26:5073-5078, 1998; Lizardi et al, Nature Genetics 19:226, 1998; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801). In some embodiments the polymerase is phi29 DNA polymerase.

In certain aspects, the nucleic acid molecule includes an amine-modified nucleotide. In such embodiments, the amine-modified nucleotide includes an acrylic acid N-hydroxysuccinimide moiety modification. Examples of other amine-modified nucleotides include, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

### Amplicon Embedding in a Tissue-Hydrogel Setting

In some embodiments, the methods disclosed include embedding one or more amplicons in the presence of hydrogel subunits to form one or more hydrogel-embedded amplicons. The hydrogel-tissue chemistry described includes covalently attaching nucleic acids to in situ synthesized hydrogel for tissue clearing, enzyme diffusion, and multiple-cycle sequencing while an existing hydrogel-tissue chemistry method cannot. In some embodiments, to enable amplicon embedding in the tissue-hydrogel setting, amine-modified nucleotides are spiked into the rolling circle amplification reaction, functionalized with an acrylamide moiety using acrylic acid N-hydroxysuccinimide esters, and copolymerized with acrylamide monomers to form a hydrogel.

As used herein, the terms “hydrogel” or “hydrogel network” mean a network of polymer chains that are water-insoluble, sometimes found as a colloidal gel in which water is the dispersion medium. In other words, hydrogels are a class of polymeric materials that can absorb large amounts of water without dissolving. Hydrogels can contain over 99% water and may include natural or synthetic polymers, or a combination thereof. Hydrogels also possess a degree of flexibility very similar to natural tissue, due to their significant water content. A detailed description of suitable hydrogels may be found in published U.S. patent application 20100055733, herein specifically incorporated by reference. As used herein, the terms “hydrogel subunits” or “hydrogel precursors” mean hydrophilic monomers, prepolymers, or polymers that can be crosslinked, or “polymerized”, to form a three-dimensional (3D) hydrogel network. Without being bound by any scientific theory, it is believed that this fixation of the biological specimen in the presence of hydrogel subunits crosslinks the components of the specimen to the hydrogel subunits, thereby securing molecular components in place, preserving the tissue architecture and cell morphology.

In some embodiments, the embedding includes copolymerizing the one or more amplicons with acrylamide. As used herein, the term “copolymer” describes a polymer which contains more than one type of subunit. The term encompasses polymer which include two, three, four, five, or six types of subunits.

In certain aspects, the embedding includes clearing the one or more hydrogel-embedded amplicons wherein the target nucleic acid is substantially retained in the one or more hydrogel-embedded amplicons. In such embodiments, the clearing includes substantially removing a plurality of cellular components from the one or more hydrogel-embedded amplicons. In some other embodiments, the clearing includes substantially removing lipids and/or proteins from the one or more hydrogel-embedded amplicons. As used herein, the term “substantially” means that the original amount present in the sample before clearing has been reduced by approximately 70% or more, such as by 75% or more, such as by 80% or more, such as by 85% or more, such as by 90% or more, such as by 95% or more, such as by 99% or more, such as by 100%.

In some embodiments, clearing the hydrogel-embedded amplicons includes performing electrophoresis on the specimen. In some embodiments, the amplicons are electrophoresed using a buffer solution that includes an ionic surfactant. In some embodiments, the ionic surfactant is sodium dodecyl sulfate (SDS). In some embodiments, the specimen is electrophoresed using a voltage ranging from about 10 to about 60 volts. In some embodiments, the specimen is electrophoresed for a period of time ranging from about 15 minutes up to about 10 days. In some embodiments, the methods further involve incubating the cleared specimen in a mounting medium that has a refractive index that matches that of the cleared tissue. In some embodiments, the mounting medium increases the optical clarity of the specimen. In some embodiments, the mounting medium includes glycerol.

### Cells

Methods disclosed herein include a method for in situ gene sequencing of a target nucleic acid in a cell in an intact tissue. In certain embodiments, the cell is present in a population of cells. In certain other embodiments, the population of cells includes a plurality of cell types including, but not limited to, excitatory neurons, inhibitory neurons, and non-neuronal cells. Cells for use in the assays of the invention can be an organism, a single cell type derived from an organism, or can be a mixture of cell types. Included are naturally occurring cells and cell populations, genetically engineered cell lines, cells derived from transgenic animals, etc. Virtually any cell type and size can be accommodated. Suitable cells include bacterial, fungal, plant and animal cells. In one embodiment of the invention, the cells are mammalian cells, e.g. complex cell populations such as naturally occurring tissues, for example blood, liver, pancreas, neural tissue, bone marrow, skin, and the like. Some tissues may be disrupted into a monodisperse suspension. Alternatively, the cells may be a cultured population, e.g. a culture derived from a complex population, a culture derived from a single cell type where the cells have differentiated into multiple lineages, or where the cells are responding differentially to stimulus, and the like.

Cell types that can find use in the subject invention include stem and progenitor cells, e.g. embryonic stem cells, hematopoietic stem cells, mesenchymal stem cells, neural crest cells, etc., endothelial cells, muscle cells, myocardial, smooth and skeletal muscle cells, mesenchymal cells, epithelial cells; hematopoietic cells, such as lymphocytes, including T-cells, such as Th1 T cells, Th2 T cells, ThO T cells, cytotoxic T cells; B cells, pre-B cells, etc.; monocytes; dendritic cells; neutrophils; and macrophages; natural killer cells; mast cells, etc.; adipocytes, cells involved with particular organs, such as thymus, endocrine glands, pancreas, brain, such as neurons, glia, astrocytes, dendrocytes, etc. and genetically modified cells thereof. Hematopoietic cells may be associated with inflammatory processes, autoimmune diseases, etc., endothelial cells, smooth muscle cells, myocardial cells, etc. may be associated with cardiovascular diseases; almost any type of cell may be associated with neoplasias, such as sarcomas, carcinomas and lymphomas; liver diseases with hepatic cells; kidney diseases with kidney cells; etc.

The cells may also be transformed or neoplastic cells of different types, e.g. carcinomas of different cell origins, lymphomas of different cell types, etc. The American Type Culture Collection (Manassas, VA) has collected and makes available over 4,000 cell lines from over 150 different species, over 950 cancer cell lines including 700 human cancer cell lines. The National Cancer Institute has compiled clinical, biochemical and molecular data from a large panel of human tumor cell lines, these are available from ATCC or the NCI (Phelps et al. (1996) Journal of Cellular Biochemistry Supplement 24:32-91). Included are different cell lines derived spontaneously, or selected for desired growth or response characteristics from an individual cell line; and may include multiple cell lines derived from a similar tumor type but from distinct patients or sites.

Cells may be non-adherent, e.g. blood cells including monocytes, T cells, B-cells; tumor cells, etc., or adherent cells, e.g. epithelial cells, endothelial cells, neural cells, etc. In order to profile adherent cells, they may be dissociated from the substrate that they are adhered to, and from other cells, in a manner that maintains their ability to recognize and bind to probe molecules.

Such cells can be acquired from an individual using, e.g., a draw, a lavage, a wash, surgical dissection etc., from a variety of tissues, e.g., blood, marrow, a solid tissue (e.g., a solid tumor), ascites, by a variety of techniques that are known in the art. Cells may be obtained from fixed or unfixed, fresh or frozen, whole or disaggregated samples. Disaggregation of tissue may occur either mechanically or enzymatically using known techniques.

### Imaging

The methods disclosed include imaging the one or more hydrogel-embedded amplicons using any of a number of different types of microscopy, e.g., confocal microscopy, two-photon microscopy, light-field microscopy, intact tissue expansion microscopy, and/or CLARITY™-optimized light sheet microscopy (COLM).

Bright field microscopy is the simplest of all the optical microscopy techniques. Sample illumination is via transmitted white light, i.e., illuminated from below and observed from above. Limitations include low contrast of most biological samples and low apparent resolution due to the blur of out of focus material. The simplicity of the technique and the minimal sample preparation required are significant advantages.

In oblique illumination microscopy, the specimen is illuminated from the side. This gives the image a 3-dimensional appearance and can highlight otherwise invisible features. A more recent technique based on this method is Hoffmann's modulation contrast, a system found on inverted microscopes for use in cell culture. Though oblique illumination suffers from the same limitations as bright field microscopy (low contrast of many biological samples; low apparent resolution due to out of focus objects), it may highlight otherwise invisible structures.

Dark field microscopy is a technique for improving the contrast of unstained, transparent specimens. Dark field illumination uses a carefully aligned light source to minimize the quantity of directly-transmitted (unscattered) light entering the image plane, collecting only the light scattered by the sample. Dark field can dramatically improve image contrast (especially of transparent objects) while requiring little equipment setup or sample preparation. However, the technique suffers from low light intensity in final image of many biological samples, and continues to be affected by low apparent resolution.

Phase contrast is an optical microscopy illumination technique that converts phase shifts in light passing through a transparent specimen to brightness changes in the image. In other words, phase contrast shows differences in refractive index as difference in contrast. The phase shifts themselves are invisible to the human eye, but become visible when they are shown as brightness changes.

In differential interference contrast (DIC) microscopy, differences in optical density will show up as differences in relief. The system consists of a special prism (Nomarski prism, Wollaston prism) in the condenser that splits light in an ordinary and an extraordinary beam. The spatial difference between the two beams is minimal (less than the maximum resolution of the objective). After passage through the specimen, the beams are reunited by a similar prism in the objective. In a homogeneous specimen, there is no difference between the two beams, and no contrast is being generated. However, near a refractive boundary (e.g. a nucleus within the cytoplasm), the difference between the ordinary and the extraordinary beam will generate a relief in the image. Differential interference contrast requires a polarized light source to function; two polarizing filters have to be fitted in the light path, one below the condenser (the polarizer), and the other above the objective (the analyzer).

Another microscopic technique using interference is interference reflection microscopy (also known as reflected interference contrast, or RIC). It is used to examine the adhesion of cells to a glass surface, using polarized light of a narrow range of wavelengths to be reflected whenever there is an interface between two substances with different refractive indices. Whenever a cell is attached to the glass surface, reflected light from the glass and that from the attached cell will interfere. If there is no cell attached to the glass, there will be no interference.

A fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” refers to any microscope that uses fluorescence to generate an image, whether it is a more simple set up like an epifluorescence microscope, or a more complicated design such as a confocal microscope, which uses optical sectioning to get better resolution of the fluorescent image.

Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (i.e., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. COLM provides an alternative microscopy for fast 3D imaging of large clarified samples. COLM interrogates large immunostained tissues, permits increased speed of acquisition and results in a higher quality of generated data.

In single plane illumination microscopy (SPIM), also known as light sheet microscopy, only the fluorophores in the focal plane of the detection objective lens are illuminated. The light sheet is a beam that is collimated in one and focused in the other direction. Since no fluorophores are excited outside the detectors' focal plane, the method also provides intrinsic optical sectioning. Moreover, when compared to conventional microscopy, light sheet methods exhibit reduced photobleaching and lower phototoxicity, and often enable far more scans per specimen. By rotating the specimen, the technique can image virtually any plane with multiple views obtained from different angles. For every angle, however, only a relatively shallow section of the specimen is imaged with high resolution, whereas deeper regions appear increasingly blurred.

Super-resolution microscopy is a form of light microscopy. Due to the diffraction of light, the resolution of conventional light microscopy is limited as stated by Ernst Abbe in 1873. A good approximation of the resolution attainable is the FWHM (full width at half-maximum) of the point spread function, and a precise widefield microscope with high numerical aperture and visible light usually reaches a resolution of ˜250 nm. Super-resolution techniques allow the capture of images with a higher resolution than the diffraction limit. They fall into two broad categories, “true” super-resolution techniques, which capture information contained in evanescent waves, and “functional” super-resolution techniques, which use experimental techniques and known limitations on the matter being imaged to reconstruct a super-resolution image.

Laser microscopy uses laser illumination sources in various forms of microscopy. For instance, laser microscopy focused on biological applications uses ultrashort pulse lasers, or femtosecond lasers, in a number of techniques including nonlinear microscopy, saturation microscopy, and multiphoton fluorescence microscopy such as two-photon excitation microscopy (a fluorescence imaging technique that allows imaging of living tissue up to a very high depth, e.g. one millimeter)

In electron microscopy (EM), a beam of electrons is used to illuminate a specimen and produce a magnified image. An electron microscope has greater resolving power than a light-powered optical microscope because electrons have wavelengths about 100,000 times shorter than visible light (photons). They can achieve better than 50 pm resolution and magnifications of up to about 10,000,000× whereas ordinary, non-confocal light microscopes are limited by diffraction to about 200 nm resolution and useful magnifications below 2000×. The electron microscope uses electrostatic and electromagnetic “lenses” to control the electron beam and focus it to form an image. These lenses are analogous to but different from the glass lenses of an optical microscope that form a magnified image by focusing light on or through the specimen. Electron microscopes are used to observe a wide range of biological and inorganic specimens including microorganisms, cells, large molecules, biopsy samples, metals, and crystals. Industrially, the electron microscope is often used for quality control and failure analysis. Examples of electron microscopy include Transmission electron microscopy (TEM), Scanning electron microscopy (SEM), reflection electron microscopy (REM), Scanning transmission electron microscopy (STEM) and low-voltage electron microscopy (LVEM).

Scanning probe microscopy (SPM) is a branch of microscopy that forms images of surfaces using a physical probe that scans the specimen. An image of the surface is obtained by mechanically moving the probe in a raster scan of the specimen, line by line, and recording the probe-surface interaction as a function of position. Examples of SPM include atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM).

Intact tissue expansion microscopy (exM) enables imaging of thick preserve specimens with roughly 70 nm lateral resolution. Using ExM the optical diffraction limit is circumvented by physically expanding a biological specimen before imaging, thus bringing sub-diffraction limited structures into the size range viewable by a conventional diffraction-limited microscope. ExM can image biological specimens at the voxel rates of a diffraction limited microscope, but with the voxel sizes of a super-resolution microscope. Expanded samples are transparent, and index-matched to water, as the expanded material is >99% water. Techniques of expansion microscopy are known in the art, e.g., as disclosed in Gao et al., Q&A: Expansion Microscopy, BMC Biol. 2017; 15:50.

### Image Registration

Image registration involves aligning common features in two or more images, which may be taken at different times, at different viewing angles, or using different color channels. Image registration is used to combine in situ sequencing imaging data from multiple images. For in situ sequencing of nucleic acids in tissue samples, images are typically taken at multiple timepoints across multiple color channels at each time point. A tissue sample may be moved between images, and there may be slight offsets and different viewing angles between different cameras taking images. The registration procedure finds common features in images and performs “micro-alignments” so that the images are aligned for downstream analysis.

In some embodiments, a common imaging dye is used at each imaging timepoint to provide rough alignment of images for each time point. This method can be used across both sequential and combinatorial modalities. For combinatorial sequencing, images across both time and channels are aligned so that the amplicon features can be accurately measured across different rounds and different channels. In some embodiments, a two-step registration process is used for image registration comprising an intra-channel registration step followed by an inter-channel registration step. For intra-channel registration, each channel is registered in three-dimensions across rounds, independently of all other channels. The overlap in amplicon features can be used to perform intra-channel registration at a sub-pixel level. Next, inter-channel registration is performed for three-dimensional registration of channels to each other. This step begins by performing an across roundmax-projection on the intra-channel registered data of the previous step. The result is a per-channel, aligned image of all amplicons which appear in a channel. The final output combines the results of both the intra-channel registration and inter-channel registration steps and applies a final per time point, per channel registration.

### Segmentation of an Image of Tissue

Any suitable method known in the art can be used for image segmentation, which involves the identification of the boundaries of individual cells in an image. Automatic or semiautomatic image analysis methods may be used for image segmentation. For example, staining of cell-membrane markers and DNA may be used to identify cells and nuclei in images, respectively. In some embodiments, conventional thresholding and watershed segmentation are used for identification of single cells in images. In some embodiments, a supervised classifier is used to automate identification of single cells in images. However, fully automatic segmentation sometimes yields poor results, especially for complicated images. Various factors can complicate image analysis, including noise, autofluorescence, low resolution, blur, unstable brightness, overlapping targets, unclear boundaries, deformation, etc. In some cases, human intervention may be needed to accurately identify separate cells in an image. In cases in which a classifier is insufficient to automatically identify single cells accurately, a human may outline at least some of the single-cells in an image to produce a set of single cells that can be used to train machine learning algorithms. Various software programs are currently available for image segmentation, including, but not limited to, Cellpose, which uses a deep learning-based segmentation method (Stringer et al. (2021) Nature Methods 18:100-106; herein incorporated by reference in its entirety), the Ilastik Toolkit, which uses a random forest classifier for cell segmentation, DeepCell, which uses a deep-learning algorithm utilizing deep convolutional neural networks for cell segmentation, Open Segmentation Framework (OpSeF), which semi-automates image segmentation using deep learning convolutional neural networks with the user manually providing some training data, CellSeg, which uses a mask region-convolutional neural network (R-CNN) for image segmentation, CODEX image processing pipeline software, which uses reference cellular markers, a reference nuclear stain, and a reference membrane stain to aid image segmentation, and CellProfiler, which uses conventional thresholding to classify a pixel as foreground if it is brighter than a certain “threshold” intensity value (cells appear as bright objects on a dark background in fluorescent microscopy images), illumination correction, declustering, and watershed segmentation to identify cells in images. For a description of image segmentation techniques and software, see, e.g., Kreshuk et al. (2019) Methods Mol. Biol. 2040:449-463, Kreshuk et al. (2014) PLOS One 9(2):e87351, David A. Van Valen et al. (2016) PLOS Comput. Biol. 12(11):e1005177, Dobson et al. (2021) Curr. Protoc. 1(5):e89, Stirling et al. (2021) BMC Bioinformatics 22(1):433, Soliman (2015) Biol Proced Online 17:11, Schapiro et al. (2017) Nat. Methods 14:873-876, Ljosa et al. (2009) PLOS Comput. Biol. 5(12):e1000603, and Lee et al. (2022) BMC Bioinformatics 23(1):46; herein incorporated by reference.

The imaging data may be chunked and distributed across many graphics processing units (GPUs) to increase the speed of image segmentation. After imaging data is chunked for image segmentation, the imaging data is stitched back together for further processing. In some cases, segmentation at chunk boundaries is problematical; therefore, each chunk at a chunk boundary may be augmented with data from its neighbors prior to processing using a distributed stitching segmentation algorithm, as described herein (see Examples). Segmentation information from a chunk is passed to a neighboring chunk at each step of the process. The first step of the process is to run the segmentation algorithm over a single chunk, augmented at the boundaries to ensure accurate segmentation will occur over the entire volume of the non-augmented area. Subsequent steps of the algorithm then take the previous results and share information with neighboring chunks. These steps then allow the chunk to update its own segmentation so that its segments at the border are stitched correctly to neighbors. For compact cells/nuclei, generally a two-step process is enough to accurately stitch together cells that span chunk boundaries.

In certain embodiments, further image processing may be performed after segmentation such as filtering image segments to remove artifactual cell-like objects, including, but not limited to, cellular debris misidentified as cells, adjacent cells merged in the same image segment, and auto-fluorescent non-cell objects.

### Devices and Systems for Performing In Situ Sequencing

Also included are devices for performing aspects of the subject methods. The subject devices may include, for example, imaging chambers, electrophoresis apparatus, flow chambers, microscopes, needles, tubing, pumps.

The present disclosure also provides systems for performing in situ sequencing. Systems may include, e.g. a power supply, a refrigeration unit, waste, a heating unit, a pump, etc. Systems may also include any of the reagents described herein, e.g. imaging buffer, wash buffer, strip buffer, Nissl and DAPI solutions. Systems in accordance with certain embodiments may also include a microscope and/or related imaging equipment, e.g., camera components, digital imaging components and/or image capturing equipment, computer processors configured to collect images according to one or more user inputs, and the like.

As discussed above, the systems described herein include a fluidics device having an imaging chamber and a pump; and a processor unit configured to perform the methods for in situ gene sequencing described herein. In some embodiments, the system enables the automation of in situ sequencing, as described herein, including, but not limited to, (a) contacting a target nucleic acid with a read oligonucleotide and a set of fluorescently labeled decoding probes, wherein the read oligonucleotide comprises a first complementarity region that is complementary to a reading sequence on the target nucleic acid, and wherein each decoding probe comprises a second complementarity region that is complementary to a probe binding site on the target nucleic acid; (b) ligating the read oligonucleotide to one of the decoding probes of the set of fluorescently labeled decoding probes to generate a fluorescent ligation product, wherein the ligation only occurs when the read oligonucleotide and the decoding probe bind to adjacent sequences on the target nucleic acid and both the read oligonucleotide and the decoding probe have sequences that are exactly complementary to the sequence of the target nucleic acid; (c) removing unligated probes; (d) imaging the fluorescent ligation product to detect the fluorescent label of the decoding probe that ligated to the read oligonucleotide, wherein the fluorescent label identifies a nucleotide of the sequence of the target nucleic acid; and (e) removing the fluorescent ligation product from the target nucleic acid by binding a competitor oligonucleotide to the target nucleic acid, wherein the competitor oligonucleotide comprises a third complementarity region comprising a sequence that is complementary to the reading sequence on the target nucleic acid, wherein the fluorescent ligation product dissociates from the target nucleic acid. In some embodiments, the method comprises: (a) contacting a fixed and permeabilized intact tissue with at least a pair of oligonucleotide primers under conditions to allow for specific hybridization, wherein the pair of primers comprise a first oligonucleotide and a second oligonucleotide; wherein each of the first oligonucleotide and the second oligonucleotide comprises a first complementarity region, a second complementarity region sequence, and a third complementarity region; wherein the second oligonucleotide further comprises a barcode sequence; wherein the first complementarity region of the first oligonucleotide is complementary to a first portion of the target nucleic acid, wherein the second complementarity region of the first oligonucleotide is complementary to the first complementarity region of the second oligonucleotide, wherein the third complementarity region of the first oligonucleotide is complementary to the third complementarity region of the second oligonucleotide, wherein the second complementary region of the second oligonucleotide is complementary to a second portion of the target nucleic acid, wherein the first portion of the target nucleic is adjacent to the second portion of the target nucleic acid; (b) adding ligase to ligate the second oligonucleotide and generate a closed nucleic acid circle; (c) performing rolling circle amplification in the presence of a nucleic acid molecule, wherein the performing comprises using the second oligonucleotide as a template and the first oligonucleotide as a primer for a polymerase to form one or more amplicons; (d) embedding the one or more amplicons in the presence of hydrogel subunits to form one or more hydrogel-embedded amplicons; and e) sequencing the one or more amplicons according to a method described herein.

In some embodiments, the system includes an imaging chamber for flowing sequencing chemicals involved in in situ DNA sequencing over a sample. In some embodiments, the system of fluidics and pumps control sequencing chemical delivery to the sample. Buffers may be added/removed/recirculated/replaced by the use of the one or more ports and optionally, tubing, pumps, valves, or any other suitable fluid handling and/or fluid manipulation equipment, for example, tubing that is removably attached or permanently attached to one or more components of a device. For example, a first tube having a first and second end may be attached to a first port and a second tube having a first and second end may be attached to a second port, where the first end of the first tube is attached to the first port and the second end of the first tube is operably linked to a receptacle, e.g. a cooling unit, heating unit, filtration unit, waste receptacle, etc.; and the first end of the second tube is attached to the second port and the second end of the second tube is operably linked to a receptacle, e.g. a cooling unit, beaker on ice, filtration unit, waste receptacle, etc.

In some embodiments, the system includes a non-transitory computer-readable storage medium that has instructions, which when executed by the processor unit, cause the processor unit to control the delivery of chemicals and synchronize this process with a microscope. In some embodiments, the non-transitory computer-readable storage medium includes instructions, which when executed by the processor unit, cause the processor unit to measure an optical signal.

### Kits

Kits are also provided for carrying out the methods described herein. In some embodiments, the kit comprises software for carrying out the computer implemented methods for processing in situ sequencing imaging data, as described herein. In some embodiments, the kit may comprise a non-transitory computer-readable medium and instructions for processing in situ sequencing imaging data, as described herein. In some embodiments, the kit comprises a system comprising a processor programmed to processing in situ sequencing imaging data according to a computer implemented method described herein; and a display component for displaying information regarding the identified target nucleic acids and the spatial locations of the identified target nucleic acids within a tissue sample.

The kit may also include agents for performing image registration or image segmentation such as imaging dyes for staining nuclei (e.g., DNA dye such as DAPI), membrane markers (e.g., antibodies specific for cell markers), or other cell components.

In some embodiments, the kit further comprises a sequencer to perform situ sequencing of target nucleic acids in a tissue.

In addition, the kits may further include (in certain embodiments) instructions for practicing the subject methods. These instructions may be present in the subject kits in a variety of forms, one or more of which may be present in the kit. For example, instructions may be present as printed information on a suitable medium or substrate, e.g., a piece or pieces of paper on which the information is printed, in the packaging of the kit, in a package insert, and the like. Another form of these instructions is a computer readable medium, e.g., diskette, compact disk (CD), flash drive, and the like, on which the information has been recorded. Yet another form of these instructions that may be present is a website address which may be used via the internet to access the information at a removed site.

### Utility

The subject methods may be used for any purpose in which sequencing readout is required and may find a number of uses in the art such as in basic research, clinical diagnostics, pathology, and forensics. For example, biomedical research applications include, but are not limited to, spatially resolved gene expression analysis for fundamental biology or drug screening. Clinical diagnostics, applications include, but are not limited to, detecting gene markers such as disease, immune responses, bacterial or viral DNA/RNA for patient samples. Examples of advantages of the methods described herein include efficiency, where it takes merely 3 or 4 days to obtain final data from a raw sample, providing speeds much faster than existing microarray or sequencing technology; highly multiplexed (up to 1000 genes); single-cell and single-molecule sensitivity; preserved tissue morphology; and/or high signal-to-noise ratio with low error rates.

In certain aspects, the subject methods may be applied to the study of molecular-defined cell types and activity-regulated gene expression in mouse visual cortex, and to be scalable to larger 3D tissue blocks to visualize short- and long-range spatial organization of cortical neurons on a volumetric scale not previously accessible. In some embodiments, the methods disclosed herein may be adapted to image DNA-conjugated antibodies for highly multiplexed protein detection.

The devices, methods, and systems of the invention can also be generalized to study a number of heterogeneous cell populations in diverse tissues. Without being bound by any scientific theory, the brain poses special challenges well suited to the sequencing methods described herein. For example, the polymorphic activity-regulated gene (ARG) expression observed across different cell types is likely to depend on both intrinsic cell-biological properties (such as signal transduction pathway-component expression), and on extrinsic properties such as neural circuit anatomy that routes external sensory information to different cells (here in visual cortex). In such cases, in situ transcriptomics can effectively link imaging-based molecular information with anatomical and activity information, thus elucidating brain function and dysfunction.

The devices, methods, and systems disclosed herein enable cellular components, e.g. lipids that normally provide structural support but that hinder visualization of subcellular proteins and molecules to be removed while preserving the 3-dimensional architecture of the cells and tissue because the sample is crosslinked to a hydrogel that physically supports the ultrastructure of the tissue. This removal renders the interior of biological specimen substantially permeable to light and/or macromolecules, allowing the interior of the specimen, e.g. cells and subcellular structures, to be microscopically visualized without time-consuming and disruptive sectioning of the tissue. The procedure is also more rapid than procedures commonly used in the art, as clearance and permeabilization, typically performed in separate steps, may be combined in a single step of removing cellular components. Additionally, the specimen can be iteratively stained, unstained, and re-stained with other reagents for comprehensive analysis. Further functionalization with the polymerizable acrylamide moiety enables amplicons to be covalently anchored within the polyacrylamide network at multiple sites.

In one example, the subject devices, methods, and systems may be employed to evaluate, diagnose or monitor a disease. “Diagnosis” as used herein generally includes a prediction of a subject's susceptibility to a disease or disorder, determination as to whether a subject is presently affected by a disease or disorder, prognosis of a subject affected by a disease or disorder (e.g., identification of cancerous states, stages of cancer, likelihood that a patient will die from the cancer), prediction of a subject's responsiveness to treatment for a disease or disorder (e.g., a positive response, a negative response, no response at all to, e.g., allogeneic hematopoietic stem cell transplantation, chemotherapy, radiation therapy, antibody therapy, small molecule compound therapy) and use of therametrics (e.g., monitoring a subject's condition to provide information as to the effect or efficacy of therapy). For example, a biopsy may be prepared from a cancerous tissue and microscopically analyzed to determine the type of cancer, the extent to which the cancer has developed, whether the cancer will be responsive to therapeutic intervention, etc.

The subject devices, methods, and systems also provide a useful technique for screening candidate therapeutic agents for their effect on a tissue or a disease. For example, a subject, e.g. a mouse, rat, dog, primate, human, etc. may be contacted with a candidate agent, an organ or a biopsy thereof may be prepared by the subject methods, and the prepared specimen microscopically analyzed for one or more cellular or tissue parameters. Parameters are quantifiable components of cells or tissues, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g., mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values. Thus, for example, one such method may include detecting cellular viability, tissue vascularization, the presence of immune cell infiltrates, efficacy in altering the progression of the disease, etc. In some embodiments, the screen includes comparing the analyzed parameter(s) to those from a control, or reference, sample, e.g., a specimen similarly prepared from a subject not contacted with the candidate agent. Candidate agents of interest for screening include known and unknown compounds that encompass numerous chemical classes, primarily organic molecules, which may include organometallic molecules, inorganic molecules, genetic sequences, etc. Candidate agents of interest for screening also include nucleic acids, for example, nucleic acids that encode siRNA, shRNA, antisense molecules, or miRNA, or nucleic acids that encode polypeptides. An important aspect of the invention is to evaluate candidate drugs, including toxicity testing; and the like. Evaluations of tissue samples using the subject methods may include, e.g., genetic, transcriptomic, genomic, proteomic, and/or metabolomics analyses.

The subject devices, methods, and systems may also be used to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, ancestry, and the like. Such detection may be used in, for example, diagnosing and monitoring disease as, e.g., described above, in personalized medicine, and in studying paternity.

A database of analytic information can be compiled. These databases may include results from known cell types, references from the analysis of cells treated under particular conditions, and the like. A data matrix may be generated, where each point of the data matrix corresponds to a readout from a cell, where data for each cell may include readouts from multiple labels. The readout may be a mean, median or the variance or other statistically or mathematically derived value associated with the measurement. The output readout information may be further refined by direct comparison with the corresponding reference readout. The absolute values obtained for each output under identical conditions will display a variability that is inherent in live biological systems and also reflects individual cellular variability as well as the variability inherent between individuals.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-54 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A computer implemented method for processing in situ sequencing imaging data, the computer performing steps comprising:
(a) receiving in situ sequencing imaging data;
(b) applying configuration parameters to the in situ sequencing imaging data, wherein the configuration parameters comprise an encoding scheme, a codebook, image acquisition parameters, and sample metadata;
(c) preprocessing imaging data by removing optical aberrations, subtracting background signal, performing deconvolution, filtering the images, and merging overlapping fields of view;
(d) performing registration of imaging data by aligning selected common features in images taken at different timepoints and across different color channels at each time point;
(e) segmenting images to determine locations of cell nuclei and cells; and
(f) performing sequential analysis of images if the in situ sequencing imaging data is from sequential sequencing, wherein said performing sequential analysis of images comprises: i) measuring an intensity of a fluorescent signal for each segmented cell and nucleus, wherein the intensity of the fluorescence signal is proportional to the amount of a target nucleic acid, and ii) subtracting estimated carry-over signal from a previous sequencing round, or
performing combinatorial analysis of images if the in situ sequencing imaging data is from combinatorial sequencing, wherein said performing sequential analysis of images comprises i) identifying each amplicon labeling a target nucleic acid from detection of barcodes, wherein invalid barcodes are removed according to their encoding scheme, ii) measuring a fluorescent signal of each amplicon at each timepoint to determine which color channel has the highest signal for each detected amplicon, closest code-book entry to a vector of signals, or clustering of pixel intensities; iii) iteratively matching signals across rounds of sequencing to membership in the code-book; iv) assigning a target identity to each detected amplicon, and v) calculating the number of each target nucleic acid present in each cell and nucleus.

2. The computer implemented method of aspect 1, wherein the in situ sequencing data are stored in a cloud data storage system.

3. The computer implemented method of aspect 2, wherein the cloud data storage system is a public cloud storage system or a private cloud storage system.

4. The computer implemented method of any one of aspects 1-3, wherein the configuration parameters are provided by a configuration file.

5. The computer implemented method of any one of aspects 1-3, wherein the configuration parameters are provided by a user inputting the configuration parameters using a management web interface.

6. The computer implemented method of any one of aspects 1-5, further comprising optimizing processing parameters by performing multiple rounds of processing of the in situ sequencing imaging data by reiterating steps (a)-(f), wherein different configuration parameters are used to process the in situ sequencing imaging data for each round of data processing.

7. The computer implemented method of any one of aspects 1-6, wherein the imaging data comprises images taken at multiple timepoints.

8. The computer implemented method of any one of aspects 1-7, wherein the imaging data comprises images taken from multiple color channels at each time point.

9. The computer implemented method of any one of aspects 1-8, wherein the imaging data further comprises morphological information, sequential readout amplicon data, or single base of combinatorial readout amplicon data.

10. The computer implemented method of any one of aspects 1-9, wherein said performing registration comprises aligning images based on detection of a common imaging dye, detectably labeled antibody, or chemical label used to stain a cell component.

11. The computer implemented method of aspect 10, wherein the common imaging dye is a fluorescent dye.

12. The computer implemented method of aspect 11, wherein the common imaging dye is a DNA dye used to stain nuclei.

13. The computer implemented method of aspect 12, wherein the DNA dye is 4′,6-diamidino-2-phenylindole (DAPI).

14. The computer implemented method of any one of aspects 1-13, wherein said performing registration comprises aligning images based on detection of immunofluorescence staining of a cell component.

15. The computer implemented method of aspect 14, wherein the cell component is a cell membrane marker.

16. The computer implemented method of any one of aspects 10-15, further comprising aligning images based on detection of fluorescently labeled amplicons.

17. The computer implemented method of aspect 10-16, further comprising aligning images from combinatorial sequencing taken at different times or from different color channels.

18. The computer implemented method of any one of aspects 1-17, wherein said performing registration comprises aligning images taken at each time point based on detection of a fluorophore used to label amplicons.

19. The computer implemented method of any one of aspects 1-18, wherein said segmenting comprises segmenting images based on detecting cell nuclei.

20. The computer implemented method of aspect 19, wherein said segmenting further comprises segmenting images based on detecting cells.

21. The computer implemented method of any one of aspects 1-20, wherein for sequential sequencing, estimating carry-over signal comprises identifying isolated amplicons for a given color channel and round of sequencing, measuring a fluorescent signal for each isolated amplicon across subsequent time points in all color channels, and selecting the isolated amplicon having the brightest fluorescent signal to measure median carry-over fraction from a given detection time point to subsequent measurement time points.

22. The computer implemented method of aspect 21, further comprising correcting fluorescent signals for a cell by calculating the true signal (S_true) based on the measured signal (S_measured) and the carry over matrix (M_co) according to the equation: S_measured=S_true*M_co.

23. The computer implemented method of any one of aspects 1-22, wherein the imaging data comprises morphology images, images from sequential sequencing, or images from combinatorial sequencing.

24. The computer implemented method of any one of aspects 1-23, wherein imaging data is divided into chunks for processing by a plurality of graphics processing units (GPUs), field-programmable gate arrays (FPGAs), or tensor processing units (TPUs).

25. The computer implemented method of aspects 24, wherein said segmenting images further comprises performing distributed stitching segmentation to stitch together cells that cross chunk boundaries.

26. The computer implemented method of any one of aspects 1-25, wherein said performing registration of imaging data from combinatorial sequencing comprises performing intra-channel registration to generate intra-channel registered data; and performing inter-channel registration on the intra-channel registered data.

27. The computer implemented method of aspect 26, wherein overlap in amplicon features is used to align images for intra-channel registration at a sub-pixel level.

28. The computer implemented method of aspect 26 or 27, wherein the inter-channel registration comprises performing a roundmax-projection on the intra-channel registered data.

29. The computer implemented method of any one of aspects 26-28, further comprising outputting aligned images of all amplicons appearing in a color channel for each channel for each time point.

30. The computer implemented method of any one of aspects 1-29, further comprising displaying information regarding the sequential analysis of images if the in situ sequencing imaging data is from sequential sequencing or displays information regarding the combinatorial analysis of images if the in situ sequencing imaging data is from combinatorial sequencing.

31. The computer implemented method of any one of aspects 1-30, further comprising displaying a target identity and target location for each identified target nucleic acid superimposed on a processed image of the tissue.

32. The computer implemented method of any one of aspects 1-31, further comprising displaying the number of each target nucleic acid present in each cell and nucleus.

33. The computer implemented method of any one of aspects 1-32, wherein said removing optical aberrations comprises removing chromatic aberrations.

34. The computer implemented method of any one of aspects 1-33, further comprising detecting amplicon locations by a method comprising:
choosing a channel with the maximum signal per round of sequencing for each amplicon;
finding barcodes of the amplicons in an encoding dictionary;
applying error correction;
performing cross-channel normalization;
summing signals per channel per round per amplicon;
performing probabilistic modeling of each amplicon based on the summing signals per channel per amplicon, background signals, and carry-over signals per sequencing round; and
decoding sources of signals using posterior likelihood probabilities with regularization or sparsity constraints.

35. The computer implemented method of any one of aspects 1-34, further comprising quantifying a gene by amplicon count by a method comprising:
performing a matching pursuit, an orthogonal matching pursuit, or a compressed sensing regime for matrix decomposition of pixel channel-round-intensity matrices into barcode-presence indicator vectors, barcode dictionary channel-round matrices, estimated background, estimated channel bleed through, and estimated channel carryover matrices;
identifying a location of an amplicon by detecting barcode membership vectors in pixel space;
performing probabilistic modeling of the image channel-round volume with both the number and x, y, and z coordinates of sources;
converting each pixel to a signal per a channel-round matrix;
assigning the corresponding barcode dictionary channel-round signal matrix entry based on a nearest neighbor; and
locating and counting assigned amplicons.

36. A non-transitory computer-readable medium comprising program instructions that, when executed by a processor in a computer, causes the processor to perform the method of any one of aspects 1-35.

37. A kit comprising the non-transitory computer-readable medium of aspect 36 and instructions for processing in situ sequencing imaging data.

38. The kit of aspect 37, further comprising agents for performing image registration or image segmentation.

39. The kit of aspect 38, wherein the agents comprise an imaging dye for staining nuclei or a detectably labeled antibody specific for a membrane marker.

40. A system comprising:
a processor programmed to process in situ sequencing imaging data of a tissue according to the computer implemented method of any one of aspects 1-35;
a display component for displaying information regarding the processed in situ sequencing imaging data; and
a storage component.

41. The system of aspect 40, wherein the processor is provided by a computer or handheld device.

42. The system of aspect 41, wherein the computer is a cloud computer.

43. The system of any one of aspects 40-42, wherein the display component displays information regarding the sequential analysis of images if the in situ sequencing imaging data is from sequential sequencing or displays information regarding the combinatorial analysis of images if the in situ sequencing imaging data is from combinatorial sequencing.

44. The system of any one of aspects 40-43, wherein the display component displays a target identity and target location for each identified target nucleic acid superimposed on a processed image of the tissue.

45. The system of any one of aspects 40-44, wherein the display component displays the number of each target nucleic acid present in each cell and nucleus.

46. The system of any one of aspects 40-45, further comprising a sequencer for performing in situ sequencing.

47 The system of any one of aspects 40-46, further comprising reagents for performing in situ sequencing.

48. The system of any one of aspects 40-47, further comprising an imaging chamber.

49. The system of any one of aspects 40-48, further comprising agents for performing image registration or image segmentation.

50. The system of aspect 49, wherein the agents comprise an imaging dye for staining nuclei or a detectably labeled antibody specific for a membrane marker.

51. The system of any one of aspects 40-50, wherein the storage component is cloud storage.

52. The system of any one of aspects 40-51, further comprising a hardware accelerator.

53. The system of any one of aspects 40-52, further comprising a plurality of graphics processing units (GPUs), field-programmable gate arrays (FPGAs), or tensor processing units (TPUs).

54. A kit comprising the system of any one of aspects 40-53 and instructions for processing in situ sequencing imaging data.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### Software for Next-Generation In Situ Sequencing

### Overview of Data Processing Software for Next-Generation In Situ Sequencing

Upon acquisition, data is streamed to cloud storage, along with a configuration file specifying the encoding scheme, codebook, and image acquisition and sample metadata. Data arriving in the cloud is entered into the cloud processing pipeline as specified by the configuration file. This custom cloud processing pipeline, is deployed on top of Kubernetes, a cloud provider-agnostic platform. Cloud data storage is a core component, and houses the raw, intermediate, and final data products. Processing begins with the upload of a dataset into the storage by the data acquisition system. Pipeline configuration parameters are set by the scientist in a data management web interface or generated automatically from a configuration file uploaded with the sequencing data. Each configuration is stored in a DataJoint cloud database; multiple configurations can be applied to a single dataset, allowing optimization of processing parameters using multiple processing runs. A set of data processing workers query the database for any outstanding work, perform the work, and inform the database as work is completed. Work requiring multiple computers is farmed out to a Dask cluster which autoscales to perform the work quickly. A single dataset can create multiple terabytes of intermediate data in cloud storage; this data can be efficiently visualized using Neuroglancer or analyzed in JupyterHub.

The image processing platform transforms raw microscope images into cell locations, 3d locations and molecular identities of amplicons via combinatorial readout, and per-cell amplicon signal via sequential readout. A round may include native or other fluorescence, morphological information, sequentially readout amplicon data, or a single base of combinatorial readout amplicon data. Data is immediately transformed by two steps: a preprocessing stage that removes chromatic aberration and performs background removal and deconvolution; and a stitching step that merges overlapping fields of view together. Once all rounds are preprocessed and stitched, two registration steps are performed in parallel: a large-scale registration using DAPI stain collected in each round, and a more precise amplicon registration using a multi-round combined fit to correct for camera offsets, residual chromatic effects, and other artefacts. Next, nuclei and cells are segmented using the morphological data (a fluorophore-labeled hybridization sequence complementary to the unique sequence of the oligo-dT label, yielding a proxy signal in the hydrogel of the total mRNA location). Amplicons are detected, decoded, and assigned to cells. In parallel, the sequential data goes through post-processing and target levels (amplicon signal of a particular encoding) are estimated per-cell.

### Advantages and Improvements Over Existing Methods, Devices or Materials.

This is the first cloud-based, fully scalable data processing pipeline for volumetric in-situ sequencing data. While several python and MATLAB libraries exist (starfish, dotdotdot, pysmFISH) to handle similar types of data, these do not automatically track the dataflow, and will not scale simultaneously analyze multiple datasets of multi-terabyte size.

The opinionated service, with a well-defined processing pipeline is designed to work with the sequencing hardware and chemistry described here, but can work with other hardware producing similar data. In exchange for its opinions, the pipeline can run in an automated fashion in a distributed cloud setting automatically.

In addition, the pipeline uses standard python data types, libraries, and file formats. This makes for a simpler learning curve, and makes the code easily accessible to data scientists.

### Processing Pipeline Stages

This section describes the actions of the individual stages of the processing, not going into details on the individual steps within a stage, or the distributed nature of the processing.

### Extract Metadata

The pipeline configuration and raw data are consumed in order to configure the downstream stages.

### Initial QA

Before any processing occurs, a subset of the data is quickly condensed in order to provide a quick assessment of the data quality.

### Preprocessing

The raw data is taken through an imperfect optical system, and two main preprocessing steps are needed. The first is correction for chromatic aberration, which fixes distortions due to the different wavelengths of the different imaging channels. The second is a deconvolution step, which increases resolution by correcting for a fixed and known “blurriness” in the microscope's optics. Additional corrections can include flat field corrections for uneven illumination and light collection and or removal of dark signal (camera biases, dark current, etc.). All image preprocessing steps are performed on the GPU.

### Stitching

Images are collected in a 2d- (or possibly 3d-) patchwork of fields of view, similar to the “Panoramic” feature of today's mobile phones. This stage utilizes Terastitcher (or optionally degrades to unblended, tiled arrangement of fields of view based on stage position information) to combine overlapping images into giant stitched images for downstream processing.

### Registration

Images are taken at multiple timepoints, and across multiple color channels at each time point. A sample may move between images, and there may be slight offsets between the different cameras taking images. The registration procedure finds common features in images and performs small “micro-alignments” so that the images are aligned for downstream analysis.

The registration has two components. The first component uses a common imaging dye used at each imaging timepoint to provide rough alignment of each time point. This first component is used across both sequential and combinatorial modalities. The second component, which is essential for the combinatorial modality, utilizes a novel procedure to simultaneously align combinatorial-based images across both time and channel. Finally, the results of the two registration components are combined and applied to the stitched images.

This stage relies primarily on the use of GPU-based cuCIM/cupy for calculations.

### Segmentation

Two morphological stains are imaged to determine where cell nuclei and cell bodies are located. The segmentation step analyzes two images with these stains and provides the location of cells and nuclei, and which pixels in the registered images belong to each cell/nucleus.

We leverage cellpose to identify nuclei and cells, and heavily make use of the dask toolkit to chunk the data and distribute this computation across hundreds of GPUs. A key algorithmic development made for the distributed nature of the pipeline was in stitching together the output of each GPU so that cells that straddled chunk boundaries were correctly composed.

### Sequential Analysis

The sequential analysis analyzes images taken in the sequential modality. Independently in each image, the amount of fluorescence signal observed with each segmented cell and nucleus is computed, which is proportional to the amount of target present.

A key development was in the handling of the signal from previous rounds “leaking” into later images. This was done by measuring the signal of isolated (not part of any cell) amplicons in the image at all time points and in all channels. By looking at the amplicon signal first detected in a roundt N and channel k, the “carry-over” into any channel at any subsequent round could be estimated and thus removed during quantification.

### Combinatorial Analysis and Segmentation Join

Analyzing data using the combinatorial modality requires simultaneous analysis of images from multiple timepoints and multiple channels. The identity of each amplicon labeling a target is determined by reading its barcode—a unique pattern of appearance in channels across rounds. For example, with 6 rounds and 4 channels, one target's barcode might be 124312, while another barcode might be 342421. To detect possible errors, invalid barcodes are removed or corrected, according to their encoding scheme—for example, under a Hamming encoding, barcodes have a minimum Hamming distance so that round errors may be corrected and multiple errors may be detected.

Each amplicon is detected in the earliest time point using the trackpy package, which provides the location and approximate size for each detection. This involves finding local maxima in the intensity data, and subsequent fitting of gaussians to peaks. Next, the signal is measured at each timepoint to determine which channel has the highest signal for each detected amplicon. The channel with the highest signal is the “call”, and a good quality call has no other channels with substantial signal. Amplicons are rejected if they have too many bad calls. Each accepted amplicon is assigned a target identity based on its error-corrected barcode.

The final output of this stage is a compilation of the number of each target present in each cell and nucleus.

### DataFlow

The data processing pipeline utilizes several technologies to run in a cloud environment and scale to process many datasets simultaneously.

### Docker

The data processing pipeline applications are containerized using Docker. Docker provides an isolated, versioned, reproducible environment for deploying the pipeline. Separate containers are produced for the processing pipeline, the web portal, and the data viewer.

### Kubernetes

Kubernetes is a provider-agnostic platform for deploying and managing containerized applications. It can run on a laptop, a local computer cluster, or on one of the public cloud offerings (e.g., Google, Amazon, Microsoft). By leveraging Kubernetes, a data processing pipeline can be easily deployed and is not locked into a single environment.

### Dask

Dask is a scalable architecture used to manage data flow across many workers. Each step within each step of the pipeline uses dask to run anywhere from 1 to 100,000 computation “tasks”. Dask manages a cluster of computer “workers” by assigning data and tasks to workers, keeping the cluster running at high efficiency (i.e. no idle workers).

### DataJoint

The DataJoint framework is used to track data flow through the various steps of the pipeline. DataJoint tracks what parameters are used to run a pipeline, and which steps need to be executed for a given data sample. It assigns pipeline steps to Dask as needed to complete the processing of a data sample. DataJoint is not designed for cloud-scale datasets, so the DataJoint functionality around data integrity is currently unused.

### Preemptibility

The computing model of the processing pipeline was chosen to scale to thousands of computers, where each computer is identical and can compute any task. This choice allows the pipeline to leverage “preemptible” cloud computing, resulting in significant (75%) savings in cost. Preemptible computers are provided at lower cost in exchange for letting the cloud provider remove the computer from use at any time. If a computer disappears, it's tasks are simply re-assigned to another computer. In practice, this results in a very small inefficiency compared to the cost savings.

### Algorithmically Novel

Several new, non-trivial algorithms used in the pipeline are described here.

### Carry-Over Correction

One issue that arises in images from the sequential modality (and to some extent in combinatorial modaility) is that the signal from a given round can still be present at a low level in subsequent rounds. This “carry-over” signal taints the measurements of later rounds and must be removed. Because a cell will have signals in many channels in many rounds, there is no straightforward way to directly tease apart the true signal.

The devised solution indirectly measures the carry-over within cells by analyzing the carry-over of isolated amplicons which should appear in a single channel of a single round. The algorithm first finds well-isolated amplicons for a given channel and round, and then measures the signal from each amplicon across subsequent time points in all channels (in case of cross-channel contamination). The brightest amplicons are selected to measure the median carry-over fraction from a given detection time point to subsequent measurement time points.

Finally, the cell signals are corrected by solving for the true signal (S_true) based on the measured signal (S_measured) and the carry over matrix (M_co):

$$S\_measured = S\_true * M\_co$$

Median isolated amplicon signals may also be used to normalize summated signals into psuedo-counts of targets.

### Distributed Stitching Segmentation

The image segmentation routines are very intensive and the data needs to be chunked and distributed across many GPUs to run quickly. In addition, segmentation algorithms will generally fail at chunk boundaries, so each chunk must be augmented with data from its neighbors prior to processing. The difficult problem arises then of how to stitch all this data back together.

The algorithm devised to tackle this problem uses a multi-step process, where segmentation information from a chunk is passed to a neighboring chunk at each step of the process. The first step of the process is to run the segmentation algorithm over a single chunk, augmented at the boundaries to ensure accurate segmentation will occur over the entire volume of the non-augmented area.

Subsequent steps of the algorithm then take the previous results and share information with neighboring chunks. These steps then allow the chunk to update its own segmentation so that its segments at the border are stitched correctly to neighbors. For compact cells/nuclei, generally a two-step process is enough to accurately stitch together cells that cross chunk boundaries.

### Multi-Channel, Multi-Round Combinatorial Registration

The combinatorial modality of data taking requires precise registration of images, as the amplicon features need to be accurately measured across many rounds and many channels. Even small registration errors can lead to data corruption or complete loss. This problem is unique to this problem space, where barcoded data is read out across rounds and channels. To solve this problem, a two step registration process leveraging cupy is used.

In the first step, intra-channel registration is performed. Each channel is registered in 3-dimensions across rounds, independently of all other channels. While the barcode patterning means a given amplicon won't be present in every round for a channel, roughly 25% of amplicons will be present in any pair of rounds. This is because the barcodes are distributed roughly randomly, and there are 4 channels. The 25% overlap in amplicon features is enough information to perform intra-channel registration at the sub-pixel level.

The second step of the registration is inter-channel registration, which performs a final per-channel 3-dimensional registration of channels to each other. This step begins by performing an across roundmax-projection on the intra-channel registered data of the previous step. The result is a per-channel, aligned image of all amplicons which appear in a channel. A small number of amplicons may be missing from some of these images—barcodes that do not contain a particular channel; but there is more than enough detail to do a final alignment.

The final output of the stage combines the results of both steps and applies the final per time point, per channel registration.

### Example 2

### Sequencing Chemistries and Encodings for Next-Generation in Situ Sequencing

### Overview

a. Sequencing by competitive annealing and ligation (SCAL)
   i. Use of competitor strands to remove signal from dynamic annealing and ligation signal addition.
      1. Design of reading sequences with competitor sequences
         a. Competitor sequences to outcompete ligation products from their labeling target
         b. Competitor sequences specific to reading sequences such that previous round signal can be removed simultaneously as current round signal is added
      2. Competitor pools to efficiently remove signal from multicolor sequential encodings or ambiguous/unknown barcode base targets
b. Reversible SCAL and SEDAL2 chemistries
   i. Forward chemistry: 5′ Phosphate on reading oligo and 5′ fluorophore on fluor oligo
   ii. Reverse chemistry: No 5′Phosphate on reading oligo; 5′ Phosphate on fluor oligo and 3′ fluorophore on fluor oligo
   iii. Barcode/encoding sequence on either or both sides of a reading sequence encoding site, maximizing efficiently readable barcode per reading sequence
   iv. Barcode distributed across one or many reading sequence sites, in forward and reverse direction, for arbitrarily long barcodes with high efficiency reads
   v. Error correcting codes such as Hamming to perform error correcting function with SCAL or SEDAL2 chemistry
c. Hyperspectral encodings
   i. Adaptation of SCAL or SEDAL2 fluor oligos to hyperspectral (more than 4 channel) regimes
d. Compressive encodings
   i. Simultaneous detection of multiple encoded positions with SCAL or SEDAL2 (on the same substrate)
      1. Compressive decoding using known codebook to demix simultaneously detected signals per amplicon
   ii. Simultaneous detection of multiple encoded signals on different substrates (compressive labeling)
      1. Non-orthogonal (per signal) encoding exploiting known orthogonality or structure in target expression pattern
   iii. Compressed sensing regime in which multiple of the same encoded signal is mixed across targets in the probe library using a known loading
      1. Subsequent compressed sensing sparse decoding from known loadings

### DETAILED DESCRIPTION

### SCAL Sequencing

In Sequencing by Competitive Annealing and Ligation (SCAL), signals are accumulated by dynamic annealing of two oligos (a reading sequencing and a fluorophore sequence) adjacently onto a substrate, subsequent ligation of the two sequences, and removal via competition by a signal-specific competitor oligo with the ligation product-substrate complex. By including a competitor-specific complementary site adjacent to the reading sequence, and by making the competitor complementary both to the competitor-specific sequence, as well as to the reading sequence and some or all of the fluorophore sequence, thermodynamic forces strongly favor competitor-product interactions, which have the effect of dissociating fluorescent ligation product oligos from their substrates and allowing for their removal from the sample by diffusive washing. Because competitor sequences have specificity to reading sequences, they can be used simultaneously with signal addition sequencing reactions for the following sequencing round. For example, while no competitor oligos are present during an initial sequencing round, subsequent sequencing reactions can include competitors for the signal of the previous round, thus dramatically reducing the time and phase complexity of each sequencing cycle. In the case of sequential encodings (see below), orthogonal reading sequences mean that competitors remove ligation products of a previous sequencing round from different substrates than those on which new signal is being accumulated by the current round. In the case of combinatorial encodings (see below), signal removal and addition occur on the same substrate. Round-specific competitor oligos have distinct competitor-specific complementary sites for each round, but share some sequence complementary to the next round's reading sequence for each combinatorial reading site (because round-to-round, signals are read out across adjacent bases, for a given reading site, see below). Thus, when a competitor oligo for a previous round is used simultaneously with signal addition for the current round, a 5-way strand interaction and competition results, which proceeds dynamically but irreversibly as new reading and fluorophore sequences are ligated together, resulting in the removal of the previous round signal from a substrate and the addition of the current round signal onto the same substrate. In some cases, SCAL is performed with competitor sequence for a previously labeled round added in a separate phase from signal addition for a current round.

### Sequencing Encoding—Sequential

Sequential encoding sequences consist of two parts: an orthogonal reading (OR) complementary sequence, which sets the round or sparsity of the read out, and the fluorophore complementary sequence, which encode the four different bases (A, T, C, and G) with four spectrally separable fluorophores (or other numbers of channels, see multi-spectral below). These two parts are placed adjacent to each other on the target, so that annealing of each of the two sequencing oligos in the presence of ligase results in a single ligation product from the two pieces. In SCAL and SEDAL2, the orthogonal reading sequences are 8-11 nucleotides (nt) long and target a melting temperature of up to 17°-20° C. in 330 mM monovalent salt. The sequences are optimized to minimize cross-hybridization between ORs and encodings for other rounds. The fluorophore complementary sequences, unlike in SEDAL sequential encoding, have distinct bases from each other for the three bases at the 3′ end, maximizing the specificity of ligation during sequencing and the hybridization specificity of the fluorophore sequence to its proper target, which has the effect of minimizing cross-talk signal in the fluorescent channels. This is especially important for sequential encoding in which signals are summed per cell as it prevents non-specific labeling signal from exceeding the noise floor.

### Sequencing Encoding—Combinatorial

In SCAL (and in improved encoding schemes for SEDAL2), combinatorial encodings are generated from error robust codebooks, such as a hamming code, subject to constraints on the desired degree of error detection and correction. For instance, a 200-gene set might use a 7-read long hamming code, with minimum hamming distance of 3, yielding one base correction and two error detections. Because sequencing uses a 2-base at a time reading scheme for added ligase specificity, each code sequenced is flanked by known bases (for example, C), such that reads of the codebook are always unambiguous. SCAL and SEDAL2 introduces a reverse chemistry mode in which reading occurs in the opposite direction (3′ to 5′ instead of 5′ to 3′), see below. This allows for the same reading sequence to be used in both forward and reverse directions. Thus, a given reading sequence can be flanked by sequence from the codebook, and codes can be separated across multiple sites. The complete code is reconstructed by reading each base in the code in a prescribed order. A read at a given code position consists of either a forward or reverse reading probe hybridizing directly adjacent to the code read position, and a fluorescently labeled dibase oligo (using, for example, the SOLID encoding scheme and containing 6 additional random N bases) hybridizing to the encoded base and ligating to the reading probe. Because read lengths in a single direction greater than three bases long fall off in efficiency due to exponentially decreasing concentrations of reading oligo for a given code (due to the ambiguous bases at the end of the reading probe), a given encoding is typically separated into islands of 3 code bases flanked by Cs. For instance, a 12-base long code would consist of C, 3 code bases, C, a reading sequence, C, 6 code bases, C, a reading sequence, C, the last 3 code bases, and C. Thus, in SCAL and SEDAL2, two reading sequence sites support a 12-base encoding, read out in two sets of forward reads of 3 bases each and two sets of reverse reads of 3 bases each. Using this encoding mechanism, codes of arbitrary length can be encoded into the oligo, where each read maintains similarly high efficiency and thus reads do not decay in quality across rounds. This allows for the use of highly error correcting codes via excess read length, genome-scale error robust coding capacities, or coding capacities for highly diverse libraries. Furthermore, the addition of multiple read sites facilitates optically sparsified encoding sets, where the total encoding across targets is bucketed, so that only a fraction of the encoded set is read out at a time (for instance, one reading site is used for half of targets and another reading site is used for the other half).

### Compressive Encoding and Decoding (Simultaneous Reads Per Amplicon)

A given code that is read out with SCAL or SEDAL2 may include multiple simultaneous reads from distinct code positions. For instance, two different bases may be read from two different read out sites, or from the same read out site in two different directions, resulting in one or two different fluorescent channels being detected for a given spot simultaneously. A naïve decoding would detect multiple signals per amplicon as a low quality read, but a decoding procedure that jointly estimates amplicon location and barcode identity, from the sparse dictionary of known codes, can be used to enable successful decoding with multiple channel labeling per amplicon (the same decoding procedure can also improve single read decoding over a naïve decoding and error correction/rejection approach). This effectively increases the coding capacity of each round from 4 to 16 channels, or correspondingly halves the number of read out rounds required of a barcode of a given length.

### Compressive Labeling (Overloaded/Multiplexed Read-Out Channels)

In cases where targets with a known expression are labeled, read out rounds may be combined to maximize the number of targets that are labeled in a given round with successful decoding. For example, if two markers strictly separate two categories of cells and can be read out in separable channels, then each category can be labeled with two simultaneous read outs for each orthogonal category. For instance, if excitatory and inhibitory cells have strictly separable markers, and can be identified as such in a given round, then other specific markers for excitatory cell subtypes can be labeled at the same time as inhibitory cell subtypes. Thus, in the ideal case, use of combinatorial marker genes or targets (forming a binary tree) can be used to create compressive sequential read out encoding schemes. This concept can additionally be extended to any structured encoding where the unit of quantification (sum per cell) has orthogonality across units.

In an alternate version of compressive labeling, target encodings (in the probe library) themselves can be overloaded into a single read out/fluorescent oligo channel according to a compressive sensing paradigm, such that a single fluorescent channel on a given round contains signal from multiple, potentially overlapping targets, and can be demixed via the known loadings in the encoding.

### Hyperspectral Encoding

Fluorescent oligos are not limited to encoding only 4 color channels. As ligase specificity extends to 2-3 bases adjacent to the ligation junction, each additional differentiating base used increases the channel coding capacity by up to 4 times. Thus, fluorescent oligos that differed from each other by 1 to 2 bases at the ligated end could be used to encode up to 16 different spectral channels; oligos that differed from each other by up to 3 bases at the end could be used to encode up to 64 different spectral channels. To achieve this many different channels, other emitters besides dyes could be used, including quantum dots or other molecules with tunable spectral signatures.

### Sequential Sequencing Oligos

Sequential (orthogonal) readout sequences (OR) were designed with sequential encodings (see above, 8-11nt orthogonal set), plus an adjacent competitor-specific complementary sequences between 2 and 16 nt in length (typically 16 nt). Competitor-specific complementary sequences do not complement the labeling target/substrate, while the read-out sequence does. Each round has both a unique competitor-specific complementary sequencing and a unique, orthogonal readout sequence. Forward chemistry sequential read outs contain a 5′-phosphate; reverse chemistry sequential read outs do not contain any modifications.

Fluorescent oligo sequences for sequential sequencing are one of 4 different fluorophore-modified oligos (in the case of 4-color imaging), that are especially distinct from each other at the end of the oligo to confer both hybridization and ligation specificity. Except in cases where sequential sequencing oligos contain “N” bases, one of four (when 4 detection color channels are used) sequential sequencing oligos typically are exactly complementary to sequence encoded on the target. When used in forward chemistry, the fluorescent oligo has a fluorophore modification at the 5′ end; when used in reverse chemistry, the fluorescent oligo sequence has a 5′ Phosphate and a fluorophore modification at the 3′ end. See hyperspectral encoding for cases in which more than 4 detection channels are used for encoding.

### Sequential Sequencing Competitors

Sequential sequencing competitor oligos were designed such that each orthogonal reading oligo has a corresponding set of competitor oligos. A corresponding set of competitor oligos complements a given orthogonal reading oligo's competitor-specific complementary sequence (2-16 nt in length, see sequential sequencing oligos), and is followed on the competitor oligo by sequence complementary to the orthogonal reading sequence (8-11 nt in length, see sequential sequencing oligos). Following this, some or all of each of the four fluorescent oligo sequence is complemented on each corresponding competitor in the competitor set for a given orthogonal reading oligo (2-8 nt). The component complementary to the fluorescent oligo maybe fully or partially complemented; partial complementarity allows higher concentration of competitor oligo to be used during the signal addition phase for the subsequent round (as fluorescent oligos are not round specific and are thus also complementary to the competitor oligo from the previous sequencing round, for example). The result is that at least one of the competitor oligo set forms a continuous or largely continuous hairpin along the length of the corresponding reading oligo—fluorescent oligo ligation product, thus displacing it from the target it was labeling previously. In the case of hyperspectral encodings, or fluorophore encodings with unknown bases (see combinatorial sequencing oligos/competitors below), the component of the competitor that is complementary to the fluorescent oligo can simply be “N” bases (a competitor pool specific to a given orthogonal reading oligo).

### Combinatorial Sequencing Oligos

Combinatorial sequencing reading oligos (RO) (which set the round/sparsity/position) of a given read are complementary to the target substrate at a particular position, such that a separate fluorescent oligo can anneal directly adjacently and be ligated together with the RO in the presence of ligase. Reading oligo sequence complementary to the target is between 8-11 nt in length. RO sequence also consists of additional competitor-specific complementary sequence (which does not complement the labeling target) adjacent to the target complementary sequence and is between 2-16 nt in length. When reading a combinatorial barcode, the first two unknown bases of the barcode (adjacent to the reading sequence) are positioned using known reading sequence bases; the third unknown base of the barcode is positioned using an additional “N” base at the end of the reading oligo, and subsequent positions (if more than 3 bases are read from a given read out site) are read via additional N bases at the reading oligo end. In the case of forward read chemistry, the reading oligo is at the 5′ end and the oligo has a 5′ phosphate modification. In the case of reverse read chemistry, the reading oligo is at the 3′ end and there is no phosphate modification.

Fluorescent oligos for combinatorial sequencing, when using 4 channel, 16-oligo SOLID encoding, consist of a pool of 16 oligos with 6 or more N bases and two known bases adjacent ot the ligation junction (at the end of the oligo), with fluorophore at the opposite end of the oligo from the known bases to set the corresponding encoding. In the case of forward read chemistry, the fluorescent oligos have a 5′ fluorescent modification and no phosphate modification; in the case of the reverse chemistry, the fluorescent oligos have a 5′ phosphate modification and a 3′ fluorescent modification.

Other fluorescent encoding schemes are also possible with this chemistry: a direct, single-base fluorescent readout is possible per channel (though conferring less specificity), or multi-base encodings with one channel per base (see hyperspectral encoding).

### Combinatorial Sequencing Competitors

Combinatorial sequencing competitors consist of a pool of competitors corresponding to each combinatorial read position. For a given position, competitor oligos contain the competitor-specific sequence complementary to the read out oligo competitor-specific sequence, as well as sequence complementary to the read out sequence itself, plus 1-5 “N” bases (typically 3) to confer additional specificity to as much of the barcode and fluorophore oligo component as possible (these bases are “N” as the barcode sequence, and thus the fluorophore oligo sequence, is not known).

What is claimed is:

1. A computer implemented method for processing in situ sequencing imaging data, the computer performing steps comprising:
(a) receiving in situ sequencing imaging data;
(b) applying configuration parameters to the in situ sequencing imaging data, wherein the configuration parameters comprise an encoding scheme, a codebook, image acquisition parameters, and sample metadata;
(c) preprocessing imaging data by removing optical aberrations, subtracting background signal, performing deconvolution, filtering the images, and merging overlapping fields of view;
(d) performing registration of imaging data by aligning selected common features in images taken at different timepoints and across different color channels at each time point;
(e) segmenting images to determine locations of cell nuclei and cells; and
(f) performing sequential analysis of images if the in situ sequencing imaging data is from sequential sequencing, wherein said performing sequential analysis of images comprises: i) measuring an intensity of a fluorescent signal for each segmented cell and nucleus, wherein the intensity of the fluorescence signal is proportional to the amount of a target nucleic acid, and ii) subtracting estimated carry-over signal from a previous sequencing round, or
performing combinatorial analysis of images if the in situ sequencing imaging data is from combinatorial sequencing, wherein said performing sequential analysis of images comprises i) identifying each amplicon labeling a target nucleic acid from detection of barcodes, wherein invalid barcodes are removed according to their encoding scheme, ii) measuring a fluorescent signal of each amplicon at each timepoint to determine which color channel has the highest signal for each detected amplicon, closest code-book entry to a vector of signals, or clustering of pixel intensities; iii) iteratively matching signals across rounds of sequencing to membership in the code-book; iv) assigning a target identity to each detected amplicon, and v) calculating the number of each target nucleic acid present in each cell and nucleus.

2. The computer implemented method of claim 1, wherein the in situ sequencing data are stored in a cloud data storage system.

3. The computer implemented method of claim 2, wherein the cloud data storage system is a public cloud storage system or a private cloud storage system.

4. The computer implemented method of any one of claims 1-3, wherein the configuration parameters are provided by a configuration file.

5. The computer implemented method of any one of claims 1-3, wherein the configuration parameters are provided by a user inputting the configuration parameters using a management web interface.

6. The computer implemented method of any one of claims 1-5, further comprising optimizing processing parameters by performing multiple rounds of processing of the in situ sequencing imaging data by reiterating steps (a)-(f), wherein different configuration parameters are used to process the in situ sequencing imaging data for each round of data processing.

7. The computer implemented method of any one of claims 1-6, wherein the imaging data comprises images taken at multiple timepoints.

8. The computer implemented method of any one of claims 1-7, wherein the imaging data comprises images taken from multiple color channels at each time point.

9. The computer implemented method of any one of claims 1-8, wherein the imaging data further comprises morphological information, sequential readout amplicon data, or single base of combinatorial readout amplicon data.

10. The computer implemented method of any one of claims 1-9, wherein said performing registration comprises aligning images based on detection of a common imaging dye, detectably labeled antibody, or chemical label used to stain a cell component.

11. The computer implemented method of claim 10, wherein the common imaging dye is a fluorescent dye.

12. The computer implemented method of claim 11, wherein the common imaging dye is a DNA dye used to stain nuclei.

13. The computer implemented method of claim 12, wherein the DNA dye is 4′,6-diamidino-2-phenylindole (DAPI).

14. The computer implemented method of any one of claims 1-13, wherein said performing registration comprises aligning images based on detection of immunofluorescence staining of a cell component.

15. The computer implemented method of claim 14, wherein the cell component is a cell membrane marker.

16. The computer implemented method of any one of claims 10-15, further comprising aligning images based on detection of fluorescently labeled amplicons.

17. The computer implemented method of claim 10-16, further comprising aligning images from combinatorial sequencing taken at different times or from different color channels.

18. The computer implemented method of any one of claims 1-17, wherein said performing registration comprises aligning images taken at each time point based on detection of a fluorophore used to label amplicons.

19. The computer implemented method of any one of claims 1-18, wherein said segmenting comprises segmenting images based on detecting cell nuclei.

20. The computer implemented method of claim 19, wherein said segmenting further comprises segmenting images based on detecting cells.

21. The computer implemented method of any one of claims 1-20, wherein for sequential sequencing, estimating carry-over signal comprises identifying isolated amplicons for a given color channel and round of sequencing, measuring a fluorescent signal for each isolated amplicon across subsequent time points in all color channels, and selecting the isolated amplicon having the brightest fluorescent signal to measure median carry-over fraction from a given detection time point to subsequent measurement time points.

22. The computer implemented method of claim 21, further comprising correcting fluorescent signals for a cell by calculating the true signal (S_true) based on the measured signal (S_measured) and the carry over matrix (M_co) according to the equation: S_measured=S_true*M_co.

23. The computer implemented method of any one of claims 1-22, wherein the imaging data comprises morphology images, images from sequential sequencing, or images from combinatorial sequencing.

24. The computer implemented method of any one of claims 1-23, wherein imaging data is divided into chunks for processing by a plurality of graphics processing units (GPUs), field-programmable gate arrays (FPGAs), or tensor processing units (TPUs).

25. The computer implemented method of claim 24, wherein said segmenting images further comprises performing distributed stitching segmentation to stitch together cells that cross chunk boundaries.

26. The computer implemented method of any one of claims 1-25, wherein said performing registration of imaging data from combinatorial sequencing comprises performing intra-channel registration to generate intra-channel registered data; and performing inter-channel registration on the intra-channel registered data.

27. The computer implemented method of claim 26, wherein overlap in amplicon features is used to align images for intra-channel registration at a sub-pixel level.

28. The computer implemented method of claim 26 or 27, wherein the inter-channel registration comprises performing a roundmax-projection on the intra-channel registered data.

29. The computer implemented method of any one of claims 26-28, further comprising outputting aligned images of all amplicons appearing in a color channel for each channel for each time point.

30. The computer implemented method of any one of claims 1-29, further comprising displaying information regarding the sequential analysis of images if the in situ sequencing imaging data is from sequential sequencing or displays information regarding the combinatorial analysis of images if the in situ sequencing imaging data is from combinatorial sequencing.

31. The computer implemented method of any one of claims 1-30, further comprising displaying a target identity and target location for each identified target nucleic acid superimposed on a processed image of the tissue.

32. The computer implemented method of any one of claims 1-31, further comprising displaying the number of each target nucleic acid present in each cell and nucleus.

33. The computer implemented method of any one of claims 1-32, wherein said removing optical aberrations comprises removing chromatic aberrations.

34. The computer implemented method of any one of claims 1-33, further comprising detecting amplicon locations by a method comprising:
choosing a channel with the maximum signal per round of sequencing for each amplicon;
finding barcodes of the amplicons in an encoding dictionary;
applying error correction;
performing cross-channel normalization;
summing signals per channel per round per amplicon;
performing probabilistic modeling of each amplicon based on the summing signals per channel per amplicon, background signals, and carry-over signals per sequencing round; and
decoding sources of signals using posterior likelihood probabilities with regularization or sparsity constraints.

35. The computer implemented method of any one of claims 1-34, further comprising quantifying a gene by amplicon count by a method comprising:
performing a matching pursuit, an orthogonal matching pursuit, or a compressed sensing regime for matrix decomposition of pixel channel-round-intensity matrices into barcode-presence indicator vectors, barcode dictionary channel-round matrices, estimated background, estimated channel bleed through, and estimated channel carryover matrices;
identifying a location of an amplicon by detecting barcode membership vectors in pixel space;
performing probabilistic modeling of the image channel-round volume with both the number and x, y, and z coordinates of sources;
converting each pixel to a signal per a channel-round matrix;
assigning the corresponding barcode dictionary channel-round signal matrix entry based on a nearest neighbor; and
locating and counting assigned amplicons.

36. A non-transitory computer-readable medium comprising program instructions that, when executed by a processor in a computer, causes the processor to perform the method of any one of claims 1-35.

37. A kit comprising the non-transitory computer-readable medium of claim 36 and instructions for processing in situ sequencing imaging data.

38. The kit of claim 37, further comprising agents for performing image registration or image segmentation.

39. The kit of claim 38, wherein the agents comprise an imaging dye for staining nuclei or a detectably labeled antibody specific for a membrane marker.

40. A system comprising:
a processor programmed to process in situ sequencing imaging data of a tissue according to the computer implemented method of any one of claims 1-35;
a display component for displaying information regarding the processed in situ sequencing imaging data; and
a storage component.

41. The system of claim 40, wherein the processor is provided by a computer or handheld device.

42. The system of claim 41, wherein the computer is a cloud computer.

43. The system of any one of claims 40-42, wherein the display component displays information regarding the sequential analysis of images if the in situ sequencing imaging data is from sequential sequencing or displays information regarding the combinatorial analysis of images if the in situ sequencing imaging data is from combinatorial sequencing.

44. The system of any one of claims 40-43, wherein the display component displays a target identity and target location for each identified target nucleic acid superimposed on a processed image of the tissue.

45. The system of any one of claims 40-44, wherein the display component displays the number of each target nucleic acid present in each cell and nucleus.

46. The system of any one of claims 40-45, further comprising a sequencer for performing in situ sequencing.

47. The system of any one of claims 40-46, further comprising reagents for performing in situ sequencing.

48. The system of any one of claims 40-47, further comprising an imaging chamber.

49. The system of any one of claims 40-48, further comprising agents for performing image registration or image segmentation.

50. The system of claim 49, wherein the agents comprise an imaging dye for staining nuclei or a detectably labeled antibody specific for a membrane marker.

51. The system of any one of claims 40-50, wherein the storage component is cloud storage.

52. The system of any one of claims 40-51, further comprising a hardware accelerator.

53. The system of any one of claims 40-52, further comprising a plurality of graphics processing units (GPUs), field-programmable gate arrays (FPGAs), or tensor processing units (TPUs).

54. A kit comprising the system of any one of claims 40-53 and instructions for processing in situ sequencing imaging data.
